(19) United States
(12) Patent Application Publication
(10) Pub. No.: US 2011/0092417 A1
(43) Pub. Date: Apr. 21, 2011

(54) LEPTIN FUSION PROTEINS

(75) Inventors: Peter Artymiuk, Sheffield (GB); Richard Ross, Sheffield (GB); Jon Sayers, Sheffield (GB)

(73) Assignee: ASTERION LIMITED, Sheffield (GB)

(21) Appl. No.: 12/671,979

(22) PCT Filed: Jul. 23, 2008
(86) PCT No.: PCT/GB08/02489

§ 371 Date: Feb. 3, 2010

Related U.S. Application Data
(60) Provisional application No. 60/956,360, filed on Aug. 16, 2007.

(30) Foreign Application Priority Data
Aug. 3, 2007 (GB) 0715216.8

(51) Int. Cl. A61K 38/22; C07K 19/00; C07H 21/00; C12N 15/63; C12N 5/10; A61P 3/04; A61P 3/10; A61P 9/00; A61P 37/06

(52) U.S. Cl. 514/5.3; 530/399; 530/324; 536/23.4; 435/320.1; 514/5.8; 435/325

(57) ABSTRACT

The disclosure relates to leptin fusion polypeptides; nucleic acid molecules encoding said polypeptides and methods of treatment that use said polypeptides.

The invention relates to leptin fusion polypeptides and dimers; nucleic acid molecules encoding said polypeptides and methods of treatment that use said polypeptides/dimers.

Cytokine receptors can be divided into three separate groups. Class 1 (referred to as the haemotopoietin or growth hormone family) receptors are characterised by four conserved cysteine residues in the amino terminal part of their extracellular domain and the presence of a conserved Trp-Ser-Xaa-Trp-Ser motif in the C-terminal part. The receptors consist of two polypeptide chains. Class I receptors can be sub-divided into the GM-CSF sub-family (which includes IL-3, IL-5, GM-CSF, GCSF) and IL-6 sub-family (which includes IL-6, IL-11 and IL-12). In the IL-6 sub-family there is a common tranduscing subunit (gp130) that associates with one or two different cytokine subunits. There is a further sub-family referred to as the IL-2 sub-family (includes IL-2, IL-4, IL-7, IL-9 and IL-15. The repeated Cys motif is also present in Class 2 (interferon receptor family) the ligands of which are α, β and γ interferons but lack the conserved Trp-Ser-Xaa-Trp-Ser motif.

Human leptin is a 16 kD protein hormone encoded by the lep gene in humans and the ob gene in mice. Leptin acts through the leptin receptor which is a single transmembrane receptor of the cytokine family. There is a single gene that encodes leptin in humans which includes three exons and two introns and spans about 18 kb of genomic DNA. Leptin links nutritional status and the immune system to control, inter alia, appetite and the immune function. The existence of mutations in either leptin or leptin receptor can result in an obese phenotype with attendant secondary symptoms associated with obesity (e.g. heart disease, diabetes type II). Leptin is mainly produced by adipose tissue in proportion to the body mass index (BMI) and, at lower levels, by organs such as the stomach and placenta. Leptin regulates body weight through inhibition of food intake and stimulation of energy expenditure. Moreover, leptin affects both the innate and adaptive immunity. On innate immunity, leptin modulates the activity of neutrophils, increases the phagocytosis of monocytes/macrophages and enhances the secretion of inflammatory mediators of the acute-phase response. On adaptive immunity, leptin promotes proliferation and interleukin 2 (IL-2) secretion by naïve T cells whereas on memory T cells, it promotes the switch towards T helper 1 (Th1) immune response by increasing interferon-γ (INF-γ) and tumor necrosis factor-α (TNF-α secretion).

If leptin expression and/or production is perturbed then the pathological manifestation of disease is complicated with effects on energy metabolism and immune status. Apart from the established linkage to obesity, reduction in leptin is associated with infertility, osteoporosis and immune suppression.

This disclosure relates to the identification of leptin recombinant forms that have improved pharmacokinetics (PK) and activity. The new leptin molecules have biological activity, form dimers and have improved stability.

According to an aspect of the invention there is provided a nucleic acid molecule comprising a nucleic acid sequence that encodes a polypeptide that has the activity of leptin comprising a leptin polypeptide linked, directly or indirectly, to at least one leptin binding domain of the leptin receptor polypeptide.

According to an aspect of the invention there is provided a fusion polypeptide comprising: the amino acid sequence of the leptin polypeptide, or active part thereof linked, directly or indirectly, to at least one leptin binding domain of the leptin receptor polypeptide.

In a preferred embodiment of the invention said fusion polypeptide comprises two leptin binding domains.

In a further preferred embodiment of the invention said fusion polypeptide comprises an immunoglobulin-like domain.

In a preferred embodiment of the invention said fusion polypeptide comprises at least one cytokine-like homology domain; preferably two cytokine-like homology domains.

In a yet further preferred embodiment of the invention said fusion polypeptide comprises at least one fibronectin III binding domain; preferably two fibronectin III binding domains.

In a preferred embodiment of the invention said fusion polypeptide comprises amino acid residues 428-535 of SEQ ID NO: 41.

In a preferred embodiment of the invention said fusion polypeptide comprises amino acid residues 536-635 of SEQ ID NO: 41.

In a preferred embodiment of the invention said fusion polypeptide comprises amino acid residues 326-437 of SEQ ID NO: 41.

In a preferred embodiment of the invention said fusion polypeptide comprises amino acid residues 62-178 of SEQ ID NO: 41.

In a preferred embodiment of the invention said fusion polypeptide comprises amino acid residues 235-325 of SEQ ID NO: 41.

In a preferred embodiment of the invention said fusion polypeptide comprises amino acid residues 639-732 of SEQ ID NO: 41.

In a preferred embodiment of the invention said fusion polypeptide comprises amino acid residues 734-829 of SEQ ID NO: 41.

In a further preferred embodiment of the invention said fusion polypeptide comprises amino acid residues 428-635 of SEQ ID NO: 41.

In a preferred embodiment of the invention said leptin polypeptide is linked to at least one leptin binding domain of leptin receptor wherein said leptin polypeptide is positioned amino-terminal to said leptin binding domain in said fusion polypeptide.

In a preferred embodiment of the invention said leptin polypeptide is linked to at least one leptin binding domain of leptin receptor wherein said leptin polypeptide is positioned carboxyl-terminal to said leptin binding domain in said fusion polypeptide.

In a preferred embodiment of the invention said leptin polypeptide is linked to at least one binding domain of the leptin receptor polypeptide by a peptide linker; preferably a flexible peptide linker.

In a preferred embodiment of the invention said peptide linking molecule comprises at least one copy of the peptide Gly Gly Gly Gly Ser.

In a preferred embodiment of the invention said peptide linking molecule comprises 2, 3, 4, 5, 6, 7, 8, 9 or 10 copies of the peptide Gly Gly Gly Gly Ser.

Preferably said peptide linking molecule consists of 6 copies of the peptide Gly Gly Gly Gly Ser.

Preferably said peptide linking molecule consists of 8 copies of the peptide Gly Gly Gly Gly Ser.

In an alternative embodiment of the invention said polypeptide does not comprise a peptide linking molecule and is a direct fusion of leptin polypeptide and at least one leptin binding domain of the leptin receptor polypeptide.

According to an aspect of the invention there is provided a nucleic acid molecule comprising a nucleic acid sequence selected from:
i) a nucleic acid sequence as represented in SEQ ID NO:3;
ii) a nucleic acid sequence as represented in SEQ ID NO:5;
iii) a nucleic acid sequence as represented in SEQ ID NO:7;
iv) a nucleic acid sequence as represented in SEQ ID NO:9;
v) a nucleic acid sequence as represented in SEQ ID NO:11;
vi) a nucleic acid sequence as represented in SEQ ID NO:13;
vii) a nucleic acid sequence as represented in SEQ ID NO:15;
viii) a nucleic acid sequence as represented in SEQ ID NO:17;
ix) a nucleic acid sequence as represented in SEQ ID NO:19;
x) a nucleic acid sequence as represented in SEQ ID NO:21;
xi) a nucleic acid sequence as represented in SEQ ID NO:23;
xii) a nucleic acid sequence as represented in SEQ ID NO:25;
xiii) a nucleic acid sequence as represented in SEQ ID NO:27;
   a nucleic acid molecule comprising a nucleic sequence that hybridizes under stringent hybridization conditions to SEQ ID NO: 3, 5, 7, 9, 11, 13, 15, 17, 19, 21, 23, 25 or 27 and which encodes a polypeptide that has leptin receptor modulating activity.

In a preferred embodiment of the invention said nucleic acid molecule encodes a leptin agonist.

In an alternative preferred embodiment of the invention said nucleic acid molecule encodes a leptin antagonist.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, N.Y., 1993). The Tₘ is the temperature at which 50% of a given strand of a nucleic acid molecule is hybridized to its complementary strand. The following is an exemplary set of hybridization conditions and is not limiting:

Very High Stringency (allows sequences that
share at least 90% identity to hybridize)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (allows sequences that share
at least 80% identity to hybridize)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Strinqency (allows sequences that share
at least 50% identity to hybridize)

| | |
|---|---|
| Hybridization: | 6x SSC at RT to 55° C. for 16-20 hours |
| Wash at least twice: | 2x-3x SSC at RT to 55° C. for 20-30 minutes each. |

In a preferred embodiment of the invention said nucleic acid molecule comprises or consists of a nucleic acid sequence as represented in SEQ ID NO:3.

In a preferred embodiment of the invention said nucleic acid molecule comprises or consists of a nucleic acid sequence as represented in SEQ ID NO: 5.

In a preferred embodiment of the invention said nucleic acid molecule comprises or consists of a nucleic acid sequence as represented in SEQ ID NO: 7.

In a preferred embodiment of the invention said nucleic acid molecule comprises or consists of a nucleic acid sequence as represented in SEQ ID NO: 9.

In a preferred embodiment of the invention said nucleic acid molecule comprises or consists of a nucleic acid sequence as represented in SEQ ID NO: 11.

In a preferred embodiment of the invention said nucleic acid molecule comprises or consists of a nucleic acid sequence as represented in SEQ ID NO: 13.

In a preferred embodiment of the invention said nucleic acid molecule comprises or consists of a nucleic acid sequence as represented in SEQ ID NO: 17.

In a preferred embodiment of the invention said nucleic acid molecule comprises or consists of a nucleic acid sequence as represented in SEQ ID NO: 19.

In a preferred embodiment of the invention said nucleic acid molecule comprises or consists of a nucleic acid sequence as represented in SEQ ID NO: 21.

In a preferred embodiment of the invention said nucleic acid molecule comprises or consists of a nucleic acid sequence as represented in SEQ ID NO: 23.

In a preferred embodiment of the invention said nucleic acid molecule comprises or consists of a nucleic acid sequence as represented in SEQ ID NO: 25.

In a preferred embodiment of the invention said nucleic acid molecule comprises or consists of a nucleic acid sequence as represented in SEQ ID NO: 27.

According to an aspect of the invention there is provided a polypeptide encoded by the nucleic acid according to the invention.

According to a further aspect of the invention there is provided a polypeptide comprising or consisting of an amino acid sequence as represented in SEQ ID NO: 4, 6, 8, 10, 12, 14, 16, 18, 20, 22, 24, 26, 28, 29, 30, 31, 32, 33, 34, 35, 36, 37, 38, 39 or 40.

According to an aspect of the invention there is provided a homodimer consisting of two polypeptides wherein each of said polypeptides comprises:
i) a first part comprising leptin, or a receptor binding domain thereof, optionally linked by a peptide linking molecule to
ii) a second part comprising at least one leptin binding domain or part thereof, of the leptin receptor.

In a preferred embodiment of the invention said homodimer comprises two polypeptides comprising or consisting of SEQ ID NO: 2, 4, 6, 8, 10, 12, 14, 16, 18, 20, 22, 24, 26, 28, 29, 30, 31, 32, 33, 34, 35, 36, 37, 38, 39 or 40.

According to a further aspect of the invention there is provided a vector comprising a nucleic acid molecule according to the invention.

In a preferred embodiment of the invention said vector is an expression vector adapted to express the nucleic acid molecule according to the invention.

A vector including nucleic acid (s) according to the invention need not include a promoter or other regulatory sequence, particularly if the vector is to be used to introduce the nucleic acid into cells for recombination into the genome for stable transfection. Preferably the nucleic acid in the vector is operably linked to an appropriate promoter or other regulatory elements for transcription in a host cell. The vector may be a bi-functional expression vector which functions in multiple hosts. By “promoter” is meant a nucleotide sequence upstream from the transcriptional initiation site and which contains all the regulatory regions required for transcription. Suitable promoters include constitutive, tissue-specific, inducible, developmental or other promoters for expression in eukaryotic or prokaryotic cells. “Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment the promoter is a constitutive, an inducible or regulatable promoter.

According to a further aspect of the invention there is provided a cell transfected or transformed with a nucleic acid molecule or vector according to the invention.

Preferably said cell is a eukaryotic cell. Alternatively said cell is a prokaryotic cell.

In a preferred embodiment of the invention said cell is selected from the group consisting of; a fungal cell (e.g. Pichia spp, Saccharomyces spp, Neurospora spp); insect cell (e.g. Spodoptera spp); a mammalian cell (e.g. COS cell, CHO cell); a plant cell.

According to a further aspect of the invention there is provided a pharmaceutical composition comprising a polypeptide according to the invention including an excipient or carrier.

In a preferred embodiment of the invention said pharmaceutical composition is combined with a further therapeutic agent.

When administered the pharmaceutical composition of the present invention is administered in pharmaceutically acceptable preparations. Such preparations may routinely contain pharmaceutically acceptable concentrations of salt, buffering agents, preservatives, compatible carriers, and optionally other therapeutic agents.

The pharmaceutical compositions of the invention can be administered by any conventional route, including injection. The administration and application may, for example, be oral, intravenous, intraperitoneal, intramuscular, intracavity, intra-articuar, subcutaneous, topical (eyes), dermal (e.g a cream lipid soluble insert into skin or mucus membrane), transdermal, or intranasal.

Pharmaceutical compositions of the invention are administered in effective amounts. An “effective amount” is that amount of pharmaceuticals/compositions that alone, or together with further doses or synergistic drugs, produces the desired response. This may involve only slowing the progression of the disease temporarily, although more preferably, it involves halting the progression of the disease permanently. This can be monitored by routine methods or can be monitored according to diagnostic methods.

The doses of the pharmaceuticals compositions administered to a subject can be chosen in accordance with different parameters, in particular in accordance with the mode of administration used and the state of the subject (i.e. age, sex). When administered, the pharmaceutical compositions of the invention are applied in pharmaceutically-acceptable amounts and in pharmaceutically-acceptable compositions.

When used in medicine salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceutical compositions may be combined, if desired, with a pharmaceutically-acceptable carrier. The term “pharmaceutically-acceptable carrier” as used herein means one or more compatible solid or liquid fillers, diluents or encapsulating substances that are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction that would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation that is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

According to a further aspect of the invention there is provided a method to treat a human subject suffering from a condition that would benefit from administration of a leptin agonist comprising administering an effective amount of at least one polypeptide according to the invention.

In a preferred method of the invention said condition is obesity.

In a further preferred method of the invention said condition is an obesity related condition.

In a preferred method of the invention said obesity related condition is type II diabetes.

In a preferred method of the invention said obesity related condition is heart disease.

In an alternative preferred method of the invention said condition is immune suppression.

According to a further aspect of the invention there is provided a method to treat a human subject suffering from a condition that would benefit from administration of a leptin antagonist comprising administering an effective amount of at least one polypeptide according to the invention.

In a preferred method of the invention said condition is anorexia.

In a further preferred method of the invention said condition is an autoimmune disease.

In a preferred method of the invention said autoimmune disease is selected from the group consisting of: multiple sclerosis, type 1 diabetes, autoimmune thyroid disease, autoimmune hepatitis, rheumatoid arthritis, autoimmune colitis, crohns disease, celiac disease, autoimmune nephritis, autoimmune neuropathy (guillan Barre), encephalopathy (Rasmussen), fibrosing alveolitis.

In a preferred method of the invention said polypeptide is administered intravenously.

In an alternative preferred method of the invention said polypeptide is administered subcutaneously.

In a further preferred method of the invention said polypeptide is administered at two day intervals; preferably said polypeptide is administered at weekly, 2 weekly or monthly intervals.

According to a further aspect of the invention there is provided a monoclonal antibody that binds the polypeptide or dimer according to the invention.

Preferably said monoclonal antibody is an antibody that binds the polypeptide or dimer but does not specifically bind leptin or leptin receptor individually.

The monoclonal antibody binds a conformational antigen presented either by the polypeptide of the invention or a dimer comprising the polypeptide of the invention.

In a further aspect of the invention there is provided a method for preparing a hybridoma cell-line producing monoclonal antibodies according to the invention comprising the steps of:
i) immunising an immunocompetent mammal with an immunogen comprising at least one polypeptide according to the invention;
ii) fusing lymphocytes of the immunised immunocompetent mammal with myeloma cells to form hybridoma cells;
iii) screening monoclonal antibodies produced by the hybridoma cells of step (ii) for binding activity to the polypeptide of (i);
iv) culturing the hybridoma cells to proliferate and/or to secrete said monoclonal antibody; and
v) recovering the monoclonal antibody from the culture supernatant.

Preferably, the said immunocompetent mammal is a mouse. Alternatively, said immunocompetent mammal is a rat.

The production of monoclonal antibodies using hybridoma cells is well-known in the art. The methods used to produce monoclonal antibodies are disclosed by Kohler and Milstein in Nature 256, 495-497 (1975) and also by Donillard and Hoffman, “Basic Facts about Hybridomas” in Compendium of Immunology V.II ed. by Schwartz, 1981, which are incorporated by reference.

According to a further aspect of the invention there is provided a hybridoma cell-line obtained or obtainable by the method according to the invention.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

Table 1 LR fusion nomenclature;

Table 2 Expression levels of the leptin LR-fusions as determined by ELISA using antibodies against the leptin receptor. (nd=not detectable)

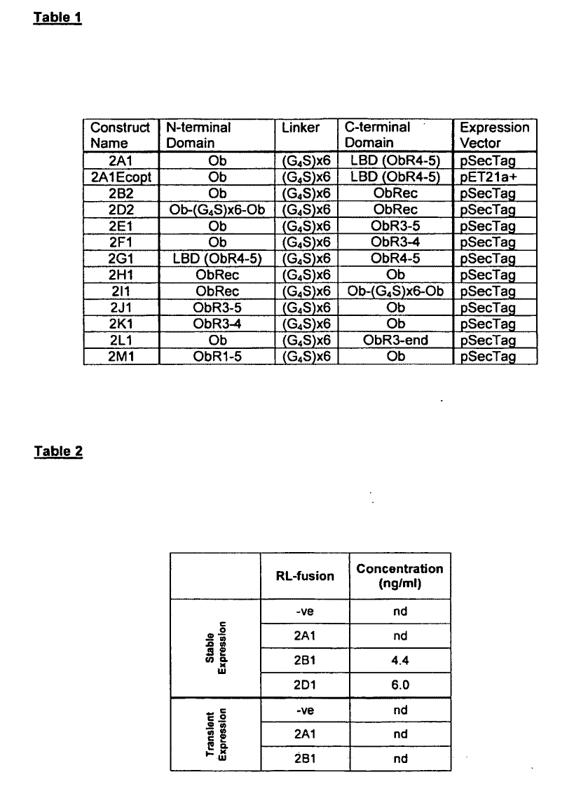

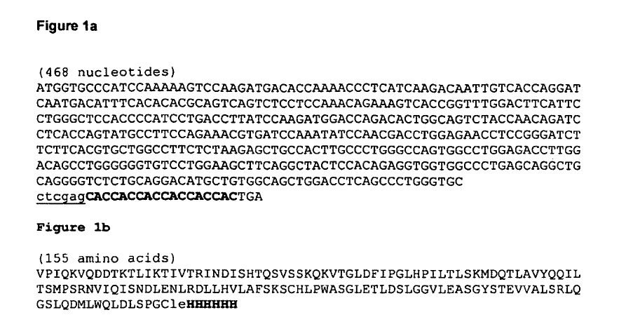

FIG. 1a is the nucleic acid sequence of bacterial expressed leptin; FIG. 1b is the amino acid sequence;

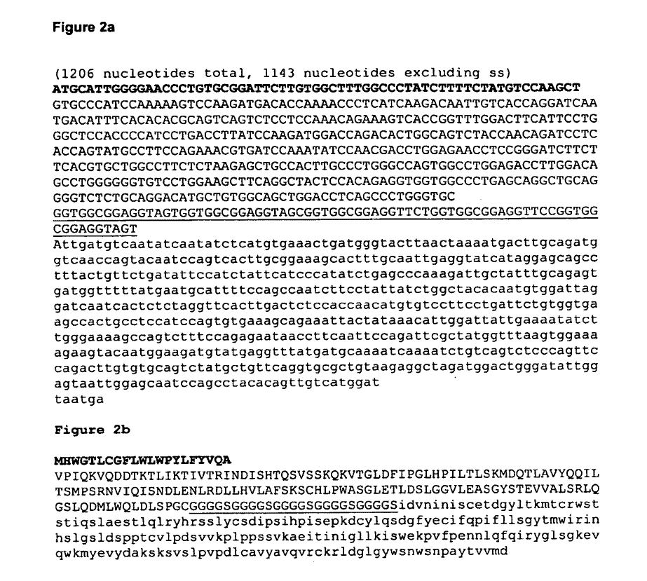

FIG. 2a is the nucleic acid sequence of LR 2A1; FIG. 2b is the amino acid sequence of LR 2A1;

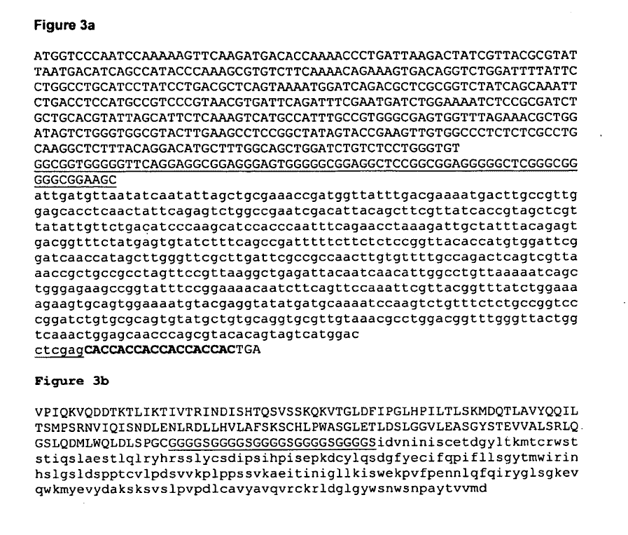

FIG. 3a is the nucleic acid sequence of LR 2A1; FIG. 2b is the amino acid sequence of LR 2A1 adapted for bacterial expression;

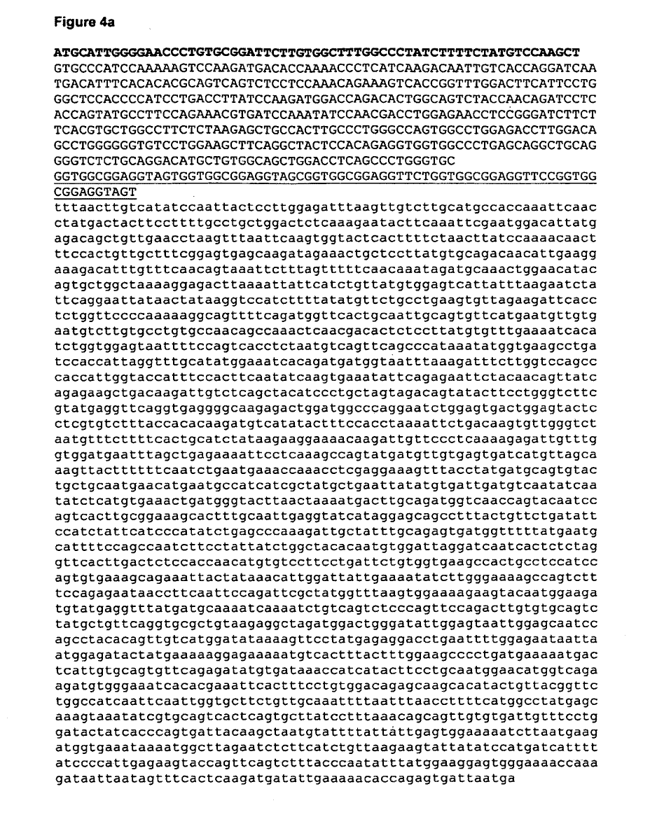

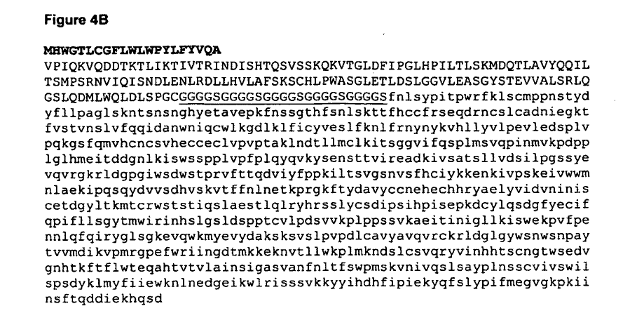

FIG. 4a is the nucleic acid sequence of LR 2B1; FIG. 2b is the amino acid sequence of LR 2B1;

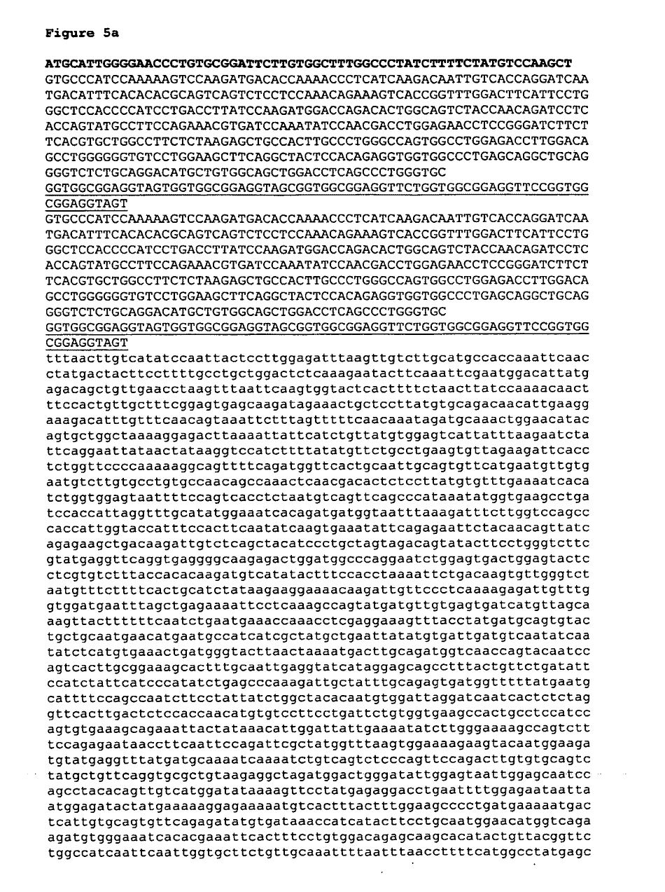

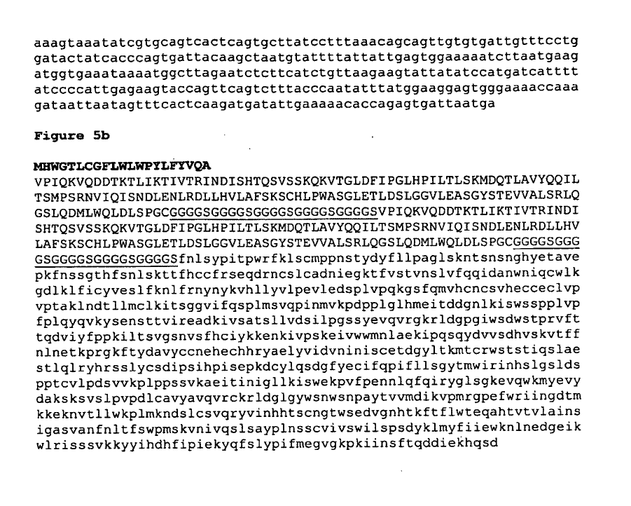

FIG. 5a is the nucleic acid sequence of LR 2D1; FIG. 2b is the amino acid sequence of LR 2D1;

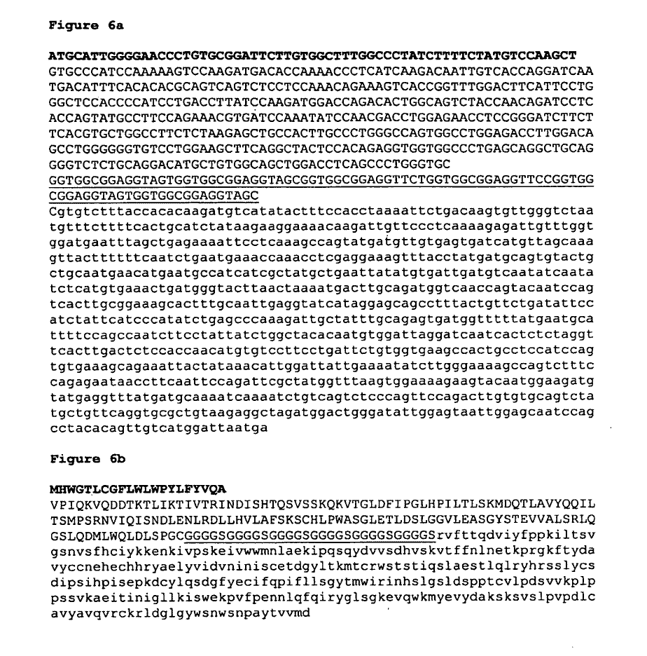

FIG. 6a is the nucleic acid sequence of LR 2E1; FIG. 2b is the amino acid sequence of LR 2E1;

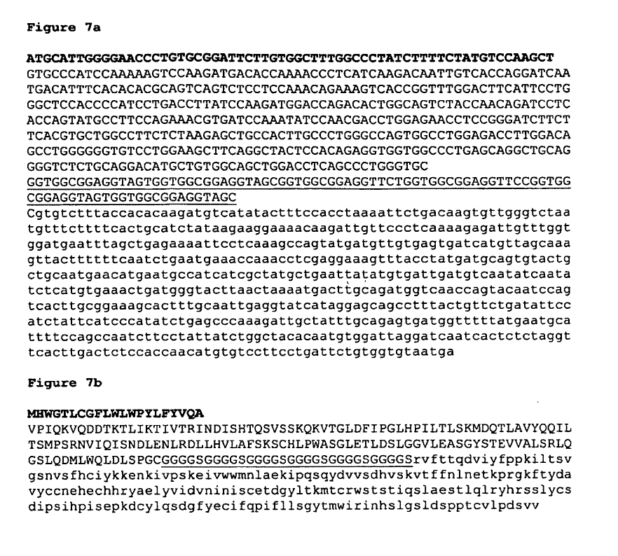

FIG. 7a is the nucleic acid sequence of LR 2F1; FIG. 2b is the amino acid sequence of LR 2F1;

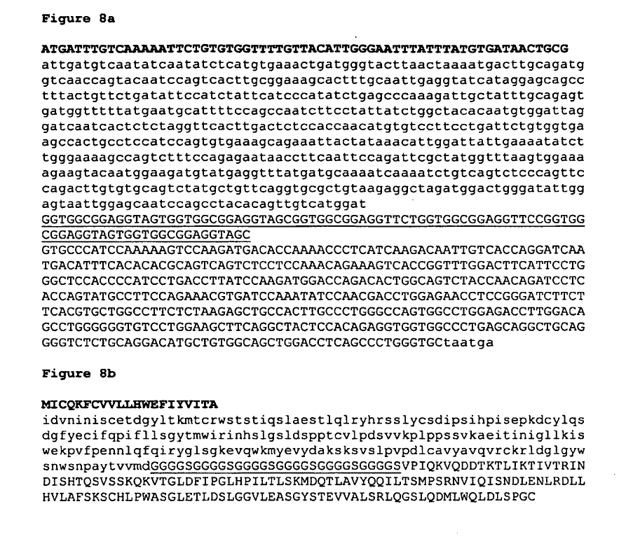

FIG. 8a is the nucleic acid sequence of LR 2G1; FIG. 2b is the amino acid sequence of LR 2G1;

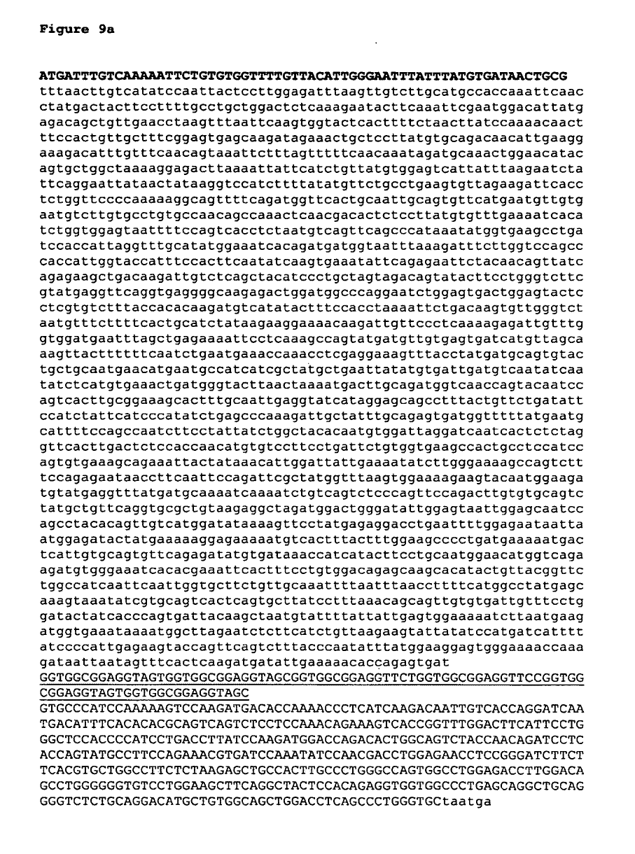

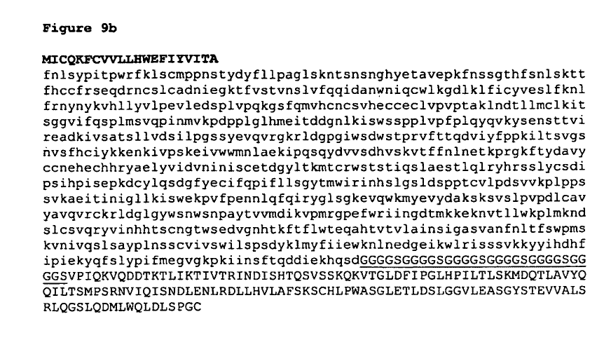

FIG. 9a is the nucleic acid sequence of LR 2H1; FIG. 2b is the amino acid sequence of LR 2H1;

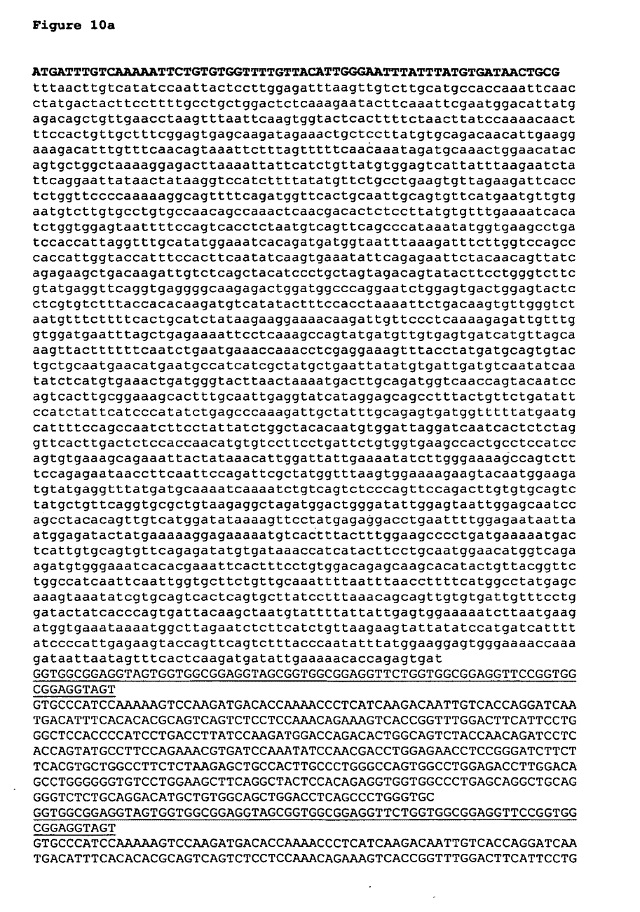

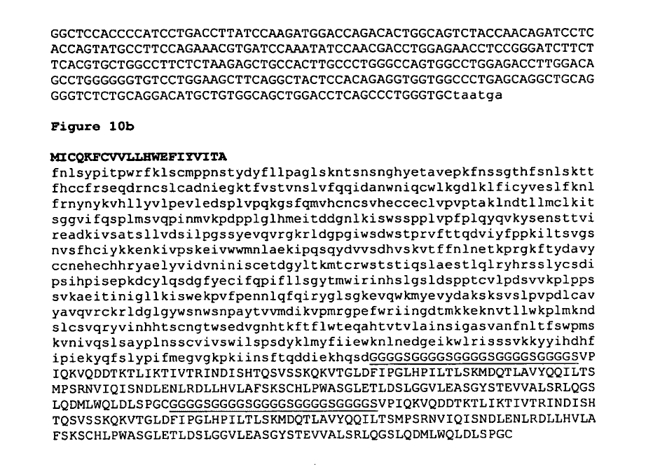

FIG. 10a is the nucleic acid sequence of LR 2I1; FIG. 2b is the amino acid sequence of LR 2I1;

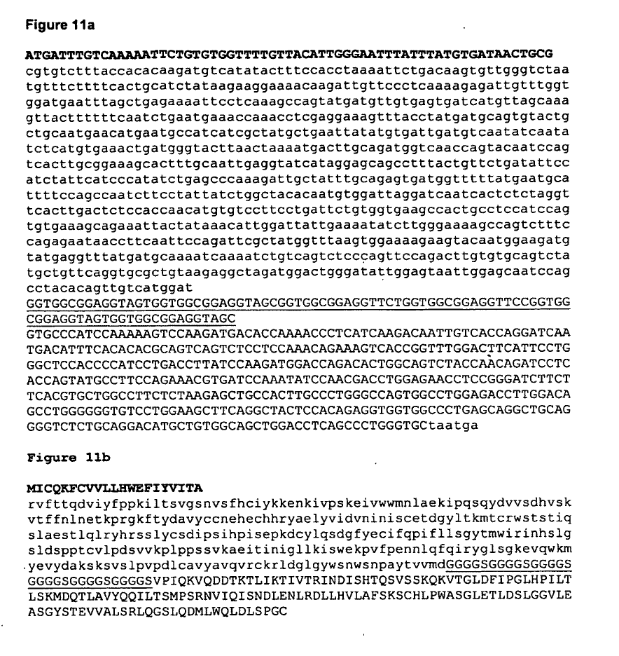

FIG. 11a is the nucleic acid sequence of LR 2J1; FIG. 2b is the amino acid sequence of LR 2J1;

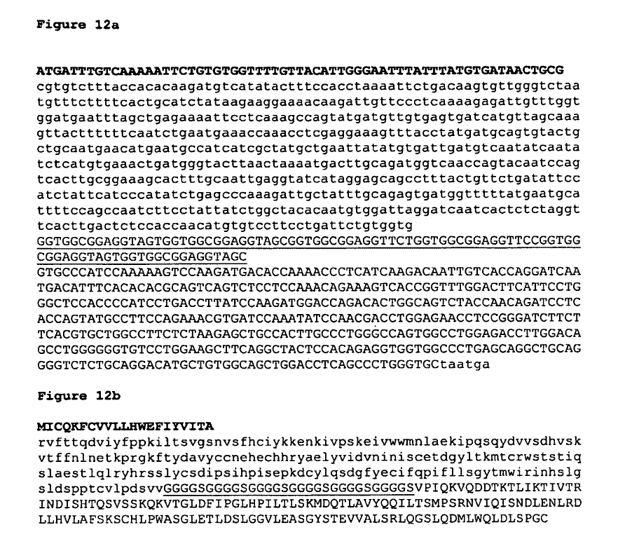

FIG. 12a is the nucleic acid sequence of LR 2K1; FIG. 2b is the amino acid sequence of LR 2K1;

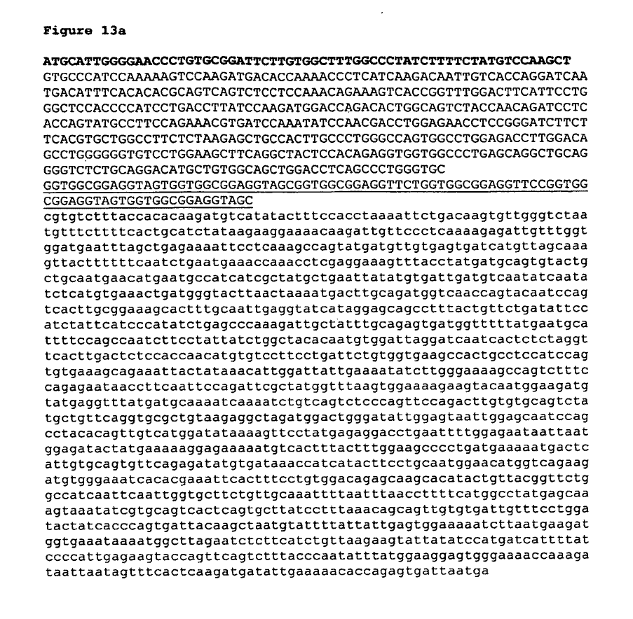

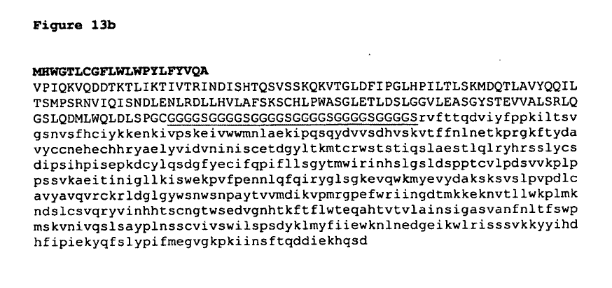

FIG. 13a is the nucleic acid sequence of LR 2L1; FIG. 2b is the amino acid sequence of LR 2L1;

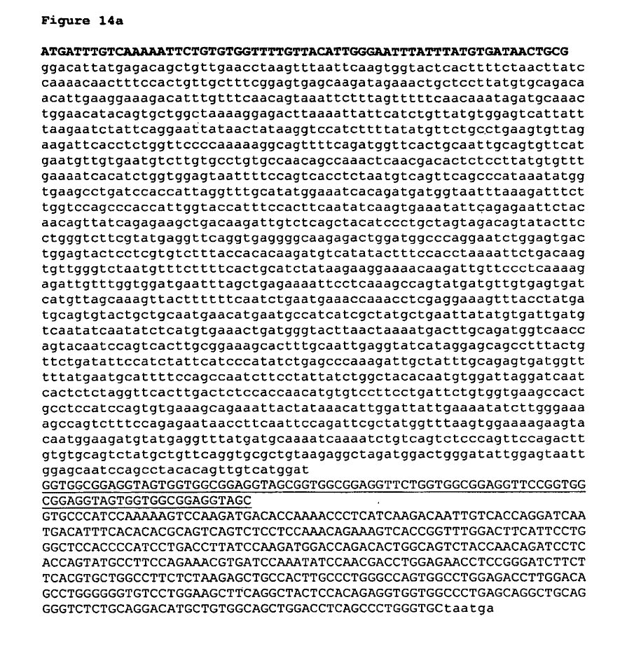

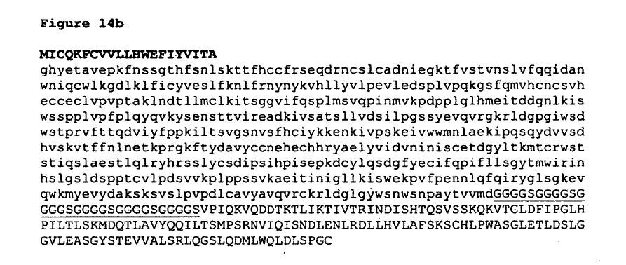

FIG. 14 is the nucleic acid sequence of LR 2M1; FIG. 2b is the amino acid sequence of LR 2M1;

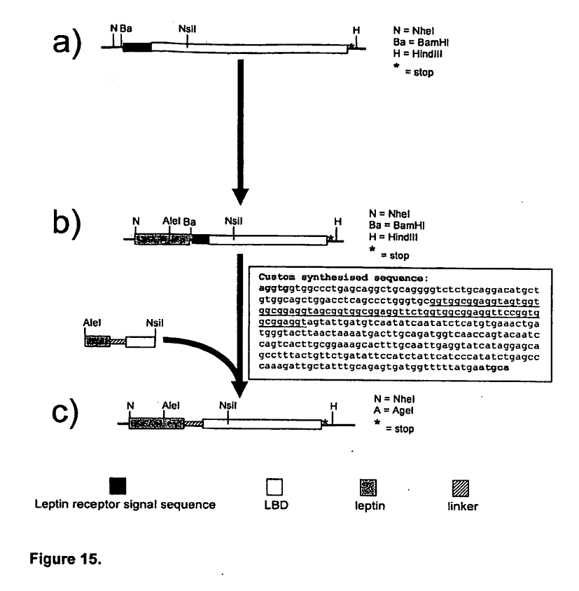

FIG. 15 a) The leptin binding domain (LBD) is ligated into the expression vector pSecTag to generate pSecTaglinkSSLBD. b) leptin is ligated into pSecTaglinkSSLBD to generate pSecTag2A1(Im). c) DNA is synthesised and ligated into pSecTag2A1(Im) to introduce a (G₄S)₅;

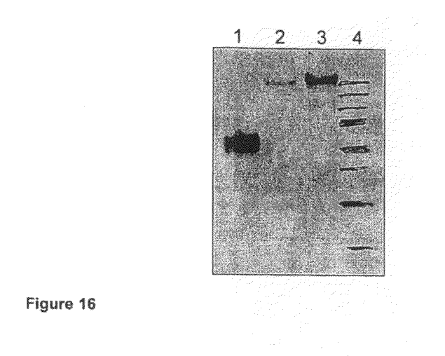

FIG. 16 Western blot of media from CHO cells expressing leptin LR-fusion. The contents of the lanes are 1 2A1 expression; 2.2B1 expression; 3 2D1 expression; 4. Markers (20, 25, 37, 50, 75, 100, 150, 200 kDa). The western blot was probed with antibodies against leptin.

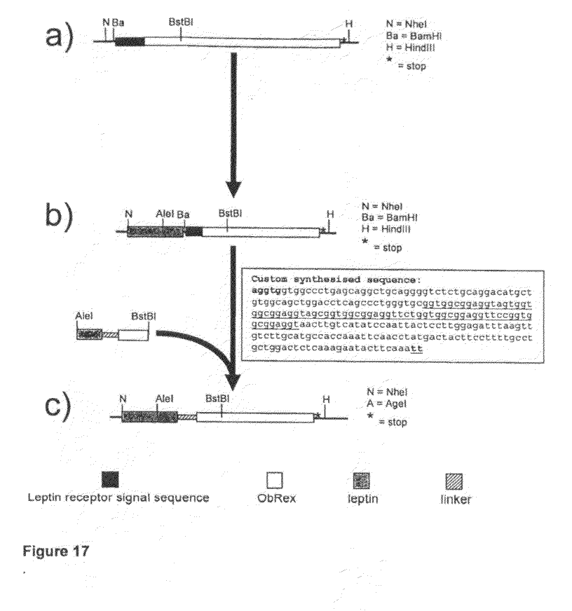

FIG. 17 a) The leptin receptor extracellular domains (ObRex) are ligated into the expression vector pSecTag to generate pSecTaglinkSSObRex. b) leptin is ligated into pSecTaglinkSSObRex to generate pSecTag2B1(Im). c) DNA is synthesised and ligated into pSecTag2B1(Im) to introduce a (G₄S)₅;

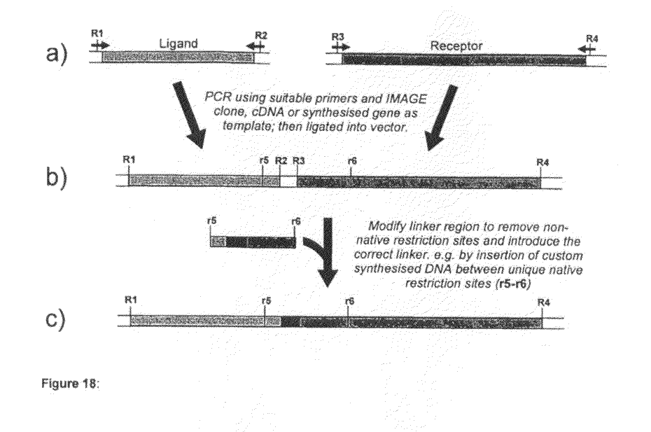

FIG. 18 a) PCR was used to generate DNA consisting of the gene of interest flanked by suitable restriction sites (contained within primers R1-4). b) The PCR products were ligated into a suitable vector either side of the linker region. c) The construct was then modified to introduce the correct linker, which did not contain any unwanted sequence (i.e. the non-native restriction sites);

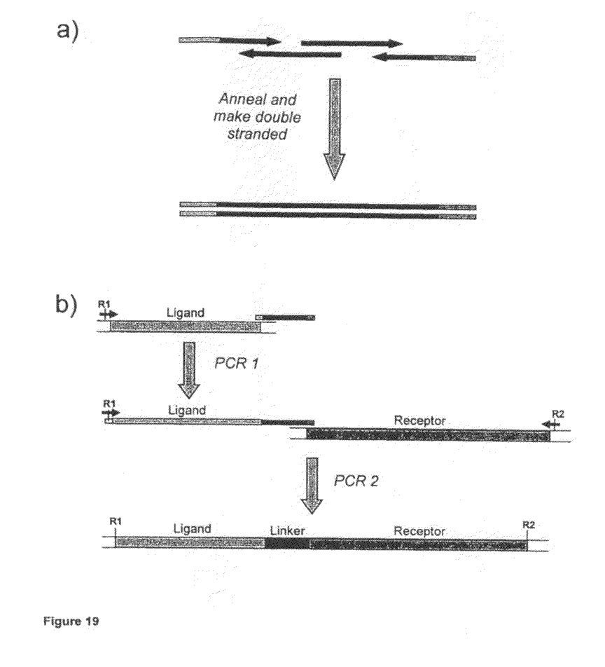

FIG. 19 a) Oligonucleotides were designed to form partially double-stranded regions with unique overlaps and, when annealed and processed would encode the linker with flanking regions which would anneal to the ligand and receptor. b) PCRs were performed using the “megaprimer” and terminal primers (R1 and R2) to produce the LR-fusion gene. The R1 and R2 primers were designed so as to introduce useful flanking restriction sites for ligation into the target vector;

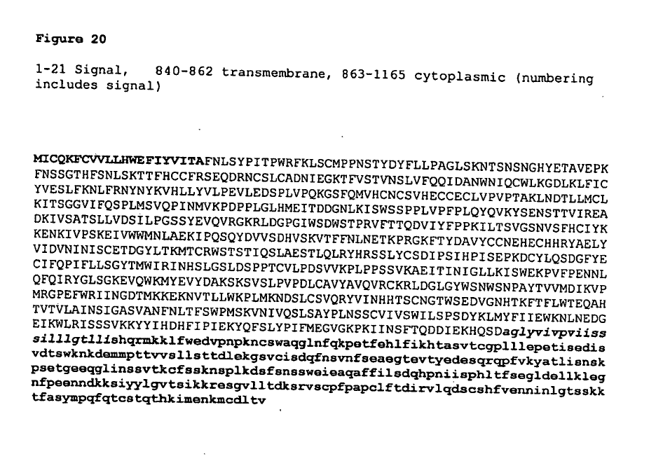

FIG. 20 is the amino acid sequence of full length leptin receptor;

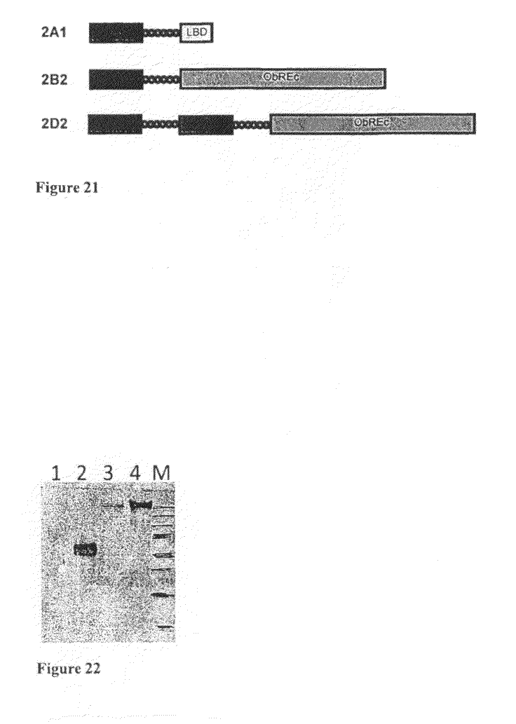

FIG. 21 is a schematic diagram of the leptin LR-fusion constructs;

FIG. 22 is an Immuno-blot analysis of CHO Flp-In stable cell lines expressing 2A1, 2B1 and 2D1constructs. Lane M=Markers (at 250, 150, 100, 75, 50, 37, 25 and 20 kDa); Lane 1=CHO Flp-In control cells, Lane 2=2A1 expression media, Lane 3=2B1 expression media, Lane 4=2D1 expression media;

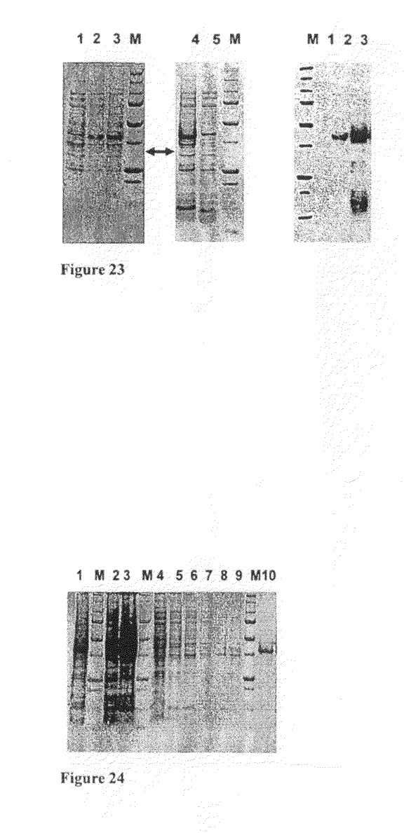

FIG. 23 is expression of 2A1Ecopt. A) Coomassie stained gels showing 2A1Ecopt expression. Lane M=Markers (at 250, 150, 100, 75, 50, 37, 25, 20 and 15 kDa); Lane 1=Expression at induction; Lane 2=Expression 4 hours post-induction; Lane 3=Expression after overnight incubation, post-induction; Lane 4=insoluble fraction of 2A1Ecopt expressing cells; Lane 5=soluble fraction of 2A1Ecopt expressing cells. B) Immuno-blot of 2A1Ecopt expression. Lane M=Markers (at 250, 150, 100, 75, 50, 37, 25, 20 and 15 kDa); Lane 1=Expression at induction; Lane 2=Expression 4 hours post-induction; Lane 3=Expression after overnight incubation, post-induction;

FIG. 24 is a Coomassie stained SDS-PAGE gel of the inclusion body prep for 2A1Ecopt. Lane M=Markers (at 250, 150, 100, 75, 50, 37, 25 and 20 kDa); Lane 1=E. coli BL21(DE3):2A1Ecopt whole cell; Lane 2=Cell lysate—soluble fraction; Lane 332 cell lysate-insoluble fraction; Lanes 4-7=2% sodium deoxychlate washes 1-4; Lanes 8-9=water washes 1-2; Lane 10=Inclusion body prep.

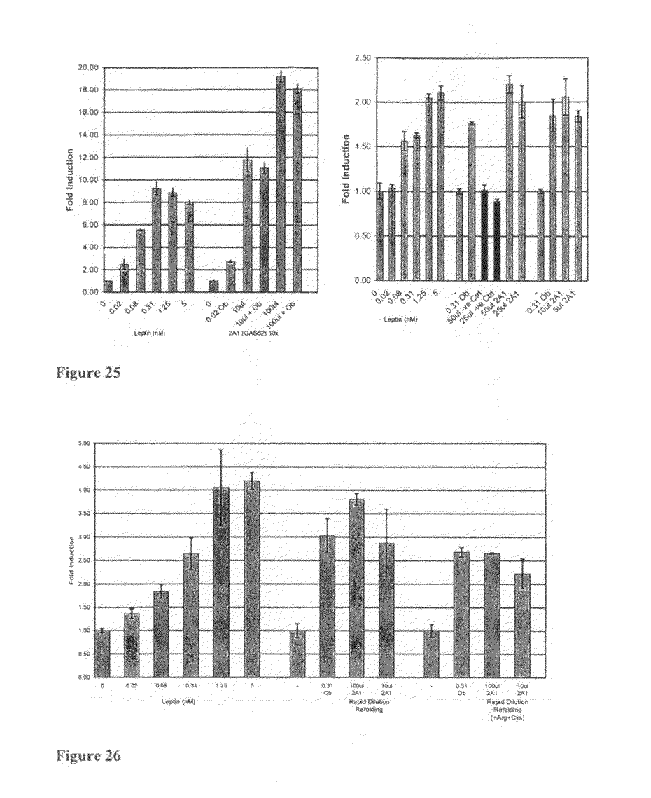

FIG. 25 is an In vitro bioassay of crude media from CHO Flp-In cells expressing 2A1. Crude media (10× concentrate) from CHO Flp-In cells expressing 2A1 was used to stimulate the cells in the leptin in vitro bioassay. The media gave agonistic activity, media from cells not expressing 2A1 gave no activity (black columns); and

FIG. 26 is an In vitro bioassay of the purified 2A1Ecopt. Refolded 2A1Ecopt samples which showed a single band at the correct size for 2A1Ecopt in the immuno-blots were used to stimulate the cells in the leptin in vitro bioassay. The samples showed agonistic activity.

### MATERIALS AND METHODS

### In vitro Testing

In vitro methods to detect and assess the activity of leptin are known in the art. For example see Liu et al (Endocrinology (1997) 138, 8: p 3548-3554); White et al (J. Biol Chemistry (1997) 272(7): p 4065-4071); and Maamra et al (Endocrinology (2007) 142(10): 4389-4393) which each describe, inter alia, the expression of leptin receptor in a cell based assay.

In addition Leptin LR-fusions were tested for in vitro activity using a dual-luciferase bioassay. Briefly, MCF-7 mammalian cells were transfected with plasmids expressing firefly luciferase induced by SIE, leptin receptor (ObR), STAT3 and Renilla luciferase (the latter three proteins are constitutively expressed). Twenty-four hours later the cells are stimulated for six hours with the leptin LR-fusion. The cells were lysed and the Firefly luciferase activity measured, this is proportional to the stimulation of the leptin receptor by the LR-fusion. Dividing this value by the activity of the constitutively expressed Renilla luciferase gives an activity corrected for experimental error.

In vivo Testing

In vivo animal models are known in the art. The ob/ob mouse model (Zhang et al Nature (1994) 372: 425-432) is homozygous for a leptin mutation and has been used to assess the activity of leptin agonists and antagonist; see Chehab et al (Nature Genetics (1996) 12: 318-320); Lord et al (Nature (1998) 394: 897-901); and Pellymounter et al (Science (1995) 269: 540-542).

### Immunological Testing

Immunoassays that measure the binding of ligand or receptor to polyclonal and monoclonal antibodies are known in the art. Commercially available antibodies are available to detect the ligand or receptor in samples and also for use in competitive inhibition studies. For example, see http://www.abcam.com/index.html, Abcam PLC.

### Recombinant Production of Fusion Proteins

The components of the fusion proteins were generated by PCR using primers designed to anneal to the ligand or receptor and to introduce suitable restriction sites for cloning into the target vector (FIG. X1a). The template for the PCR comprised the target gene and was obtained from IMAGE clones, cDNA libraries or from custom synthesised genes. Once the ligand and receptor genes with the appropriate flanking restriction sites had been synthesised, these were then ligated either side of the linker region in the target vector (FIG. X1b). The construct was then modified to contain the correct linker without flanking restriction sites by the insertion of a custom synthesised length of DNA between two unique restriction sites either side of the linker region, by mutation of the linker region by ssDNA modification techniques, by insertion of a primer duplex/multiplex between suitable restriction sites or by PCR modification (FIG. X1c).

Alternatively, the linker with flanking sequence, designed to anneal to the ligand or receptor domains of choice, was initially synthesised by creating an oligonucleotide duplex and this processed to generate double-stranded DNA (FIG. X2a). PCRs were then performed using the linker sequence as a “megaprimer”, primers designed against the opposite ends of the ligand and receptor to which the “megaprimer” anneals to and with the ligand and receptor as the templates. The terminal primers were designed with suitable restriction sites for ligation into the expression vector of choice (FIG. X2b).

### Expression and Purification of Fusion Proteins

Expression was carried out in a suitable system (e.g. mammalian CHO cells, E. coli, etc.) and this was dependant on the vector into which the LR-fusion gene was generated. Expression was then analysed using a variety of methods which could include one or more of SDS-PAGE, Native PAGE, western blotting, ELISA.

Once a suitable level of expression was achieved the RL-fusions were expressed at a larger scale to produce enough protein for purification and subsequent analysis.

Purification was carried out using a suitable combination of one or more chromatographic procedures such as ion exchange chromatography, hydrophobic interaction chromatography, ammonium sulphate precipitation, gel filtration, size exclusion and/or affinity chromatography (using nickel/cobalt-resin, antibody-immobilised resin and/or ligand/receptor-immobilised resin).

Purified protein was analysed using a variety of methods which could include one or more of Bradford's assay, SDS-PAGE, Native PAGE, western blotting, ELISA.

### Characterisation of LR-Fusions

Denaturing PAGE, native PAGE gels and western blotting were used to analyse the fusion polypeptides and western blotting performed with antibodies non-conformationally sensitive to the LR-fusion. Native solution state molecular weight information can be obtained from techniques such as size exclusion chromatography using a Superose G200 analytical column and analytical ultracentrifugation.

### Construction of LR-Fusions

The 2A1, 2B1 and 2D1 genes were synthesised by generating the Ob, LBD, ObREc and linker components with unique, compatible restriction sites at either end and ligating them together to form the complete gene. Extraneous sequence (i.e. the restriction sites) in 2B1 and 2D1 were subsequently removed by ligating in custom synthesised DNA fragments (Genecust, France) between unique restriction sites within the Ob, LBD and ObREc genes, this generated 2B2 and 2D2. The 2A1Ecopt gene was generated by custom DNA synthesis (Genecust, France) and ligated into pET21a+. The 2A1Ecopt sequence is codon optimised for expression in E. coli and has a C-terminal His tag.

### Expression of LR-Fusions

### Mammalian Expression:

Stable cell lines were generated using a modified Invitrogen vector pSecTag-V5/FRT-Hist in 6-well plates using Fugene-6 as the transfection reagent. The CHO Flp-In cells were co-transfected with the expression vector and pOG44, a plasmid that expresses flp recombinase an enzyme which causes the recombination of the LR-fusion gene into a “hot-spot” of the cell chromosome. Hygromycin B was used to select for cells with positive recombinants.

Once the stable cell lines had been established they were grown on 75 cm² culture plates, at a confluency of 50-70% the media was changed to serum free media. The cultures were incubated for a further 2-4 days after which media samples were taken. These were run on 13% SDS-PAGE gels and transferred to PVDF membrane for immuno-blotting. After blocking in 5% (w/v) milk protein in PBS+0.05% (v/v) Tween 20, sample detection was carried out using a specific anti-leptin antibody together with a Horse Radish Peroxidase (HRP) conjugated secondary antibody. Visualisation was by chemiluminescence on photographic film using an HRP detection kit.

The immuno-blots showing the expression of the LR-fusions from the mammalian system are shown in FIG. 22.

E. coli Expression:

pET21a+:2A1Ecopt was transformed into chemically competent E. coli BL21(DE3) cells. Clones expressing 2A1Ecopt were then grown in LB media supplemented with carbenicillin (100 μg/ml) and grown on a flat bed shaker at room temperature. Induction was performed with 1 mM IPTG at an OD₆₀₀ of 0.4 and the culture grown for overnight. The cells were then harvested and lysed, samples were then run on SDS-PAGE gels and coomassie stained or immuno-blotted.

The immuno-blots showing the expression of the LR-fusions from the E. coli system are shown in FIG. 23.

### Purification of LR-Fusions

E. coli Expressed LR-Fusion Purification (2A1Ecopt)

2A1Ecopt was expressed in E. coli BL21(DE3) cells from the plasmid pET21a+:2A1Ecopt.

2A1Ecopt was purified using a Ni-Probond resin column. The insoluble fraction of the lysed cells was washed four times with 2% sodium deoxycholate and the two times with distilled water to give an inclusion body prep. This step removed most of the contaminating proteins giving >80% purity for 2A1Ecopt; FIG. 24.

### Statistics

Two groups were compared with a Student's test if their variance was normally distributed or by a Student-Satterthwaite's test if not normally distributed. Distribution was tested with an F test. One-way ANOVA was used to compare the means of 3 or more groups and if the level of significance was p<0.05 individual comparisons were performed with Dunnett's tests. All statistical tests were two-sided at the 5% level of significance and no imputation was made for missing values.

### Example 1

### 2A1

DNA encoding the leptin binding domain (LBD) domains of the leptin receptor (ObR) flanked by BamHI and HindIII was produced by PCR. This was then ligated into a modified pSecTag-FRT-V5-His TOPO vector to produce pSecTagLinkSSLBD (FIG. 15a). DNA encoding leptin flanked by NheI and BamHI was produced by PCR using the primers; NheObssF (5′-gggaaagctagccaccatgcattggggaaccctgtgcg-3′) and ObBamR (5′-gggaaaggatccgcacccagggctgaggtcc-3′). This was ligated into pSecTagLinkSSLBD to produce pSecTag2A1(Im) (FIG. 15b). The linker region was custom synthesised between AleI and NsiI restriction sites and this inserted into pSecTag2A1(Im) to give pSecTag2A1stop (FIG. 15c). pSecTag2A1stop was transfected into Chinese Hamster Ovary (CHO) cells and transient and stable expression cell lines developed. However western blot of the expression media, using antibodies against leptin, showed 2A1 was expressed (FIG. 16). 2A1 was also expressed in Escherichia coli cells. The amino acid sequence for 2A1 was back-translated with optimisation for E. coli codon usage. The codon optimised gene (2A1Ecopt) was custom gene synthesised and then inserted into the pET21a+expression vector and the protein expressed from E. coli BL21 (DE3) cells.

### Example 2

### 2B1

DNA encoding the leptin receptor extracellular domain (ObRex) flanked by BamHI and HindIII was produced by PCR. This was then ligated into a modified pSecTag-FRT-V5-His TOPO vector to produce pSecTagLinkSSObRex (FIG. 17a). DNA encoding leptin flanked by NheI and BamHI was produced by PCR using the primers; NheObssF (5′-gggaaagctagccaccatgcattggggaaccctgtgcg-3′) and ObBamR (5′-gggaaaggatccgcacccagggctgaggtcc-3′). This was ligated into pSecTagLinkSSObRex to produce pSecTag2B1(Im) (FIG. 17b). The linker region was custom synthesised between AleI and BstBI restriction sites and this inserted into pSecTag2B1(Im) to give pSecTag2B1stop (FIG. 17c). pSecTag2B1stop was transfected into Chinese Hamster Ovary (CHO) cells and transient and stable expression cell lines developed. Expression levels were determined to be in the low ng/ml levels as measured by ELISA using antibodies against ObR (Table 2). However these expression levels may have been underestimated by the ELISA since the western blot of the expression media suggests that higher levels of expression have been achieved (FIG. 16).

### Example 3

### 2D1

pSecTag2D1stop was synthesised in a similar way to pSecTag2B1stop, above. pSecTag2B1stop was transfected into Chinese Hamster Ovary (CHO) cells and transient and stable expression cell lines developed. Expression levels were determined to be in the low ng/ml levels as measured by ELISA using antibodies against ObR (Table 2). However these expression levels may have been underestimated by the ELISA since the western blot of the expression media suggests that higher levels of expression have been achieved (FIG. 16).

### Example 4

### In vitro Bioassay Results

The in vitro bioassay utilises a dual-luciferase reporter system to measure the activity of stimulated MCF-7 cells.

MCF-7 cells were transfected with plasmid expressing ObR, STATS, SIE and Renilla luciferase. Activation of the ObR caused an inducible, proportional expression of firefly luciferase via STAT3 and SIE; the Renilla luciferase was constitutively expressed and acted as a control to normalise the firefly luciferase activity measurement. Both the firefly and Renilla luciferase were measured using the Dual-Luciferase Reporter Assay System (Promega) and a luminometer. Dividing the firefly luciferase measurement by the Renilla luciferase measurement gave a corrected activity (i.e. correction for differences between samples such as cell density and transfection efficiency). The corrected activity was then divided by the activity of un-stimulated cells to give a “fold induction” value.

The bioactivity for 2A1 (10× concentrate) and 2A1Ecopt (purified) is shown in FIGS. 25 and 26 respectively.

1. (canceled)

2. A fusion polypeptide comprising: the amino acid sequence of the leptin polypeptide, or active part thereof linked, directly or indirectly, to at least one leptin binding domain of the leptin receptor polypeptide.

3. (canceled)

4. A fusion polypeptide according to claim 2 wherein said fusion polypeptide comprises an immunoglobulin-like domain.

5. A fusion polypeptide according to claim 2 wherein said polypeptide comprises at least one cytokine-like homology domain.

6. (canceled)

7. A fusion polypeptide according to claim 2 wherein said polypeptide comprises at least one fibronectin III binding domain.

8. (canceled)

9. A fusion polypeptide according to claim 2 wherein said polypeptide comprises a sequence selected from the group consisting of:
(a) amino acid residues 425-535 of SEQ ID NO: 41;
(b) amino acid residues 536-635 of SEQ ID NO: 41;
(c) amino acid residues 326-427 of SEQ ID NO: 41;
(d) amino acid residues 62-178 of SEQ ID NO: 41;
(e) amino acid residues 235-325 of SEQ ID NO: 41;
(f) amino acid residues 636-733 of SEQ ID NO: 41;
(g) amino acid residues 734-829 of SEQ ID NO: 41; and
(h) amino acid residues 428-635 of SEQ ID NO: 41.

10-18. (canceled)

19. A fusion polypeptide according to claim 2 wherein said leptin polypeptide is linked to at least one binding domain of the leptin receptor polypeptide by a peptide linker.

20. A fusion polypeptide according to claim 19 wherein said peptide linking molecule comprises at least one copy of the peptide Gly Gly Gly Gly Ser (SEQ ID NO: 44).

21-23. (canceled)

24. A fusion polypeptide according to claim 2 wherein said fusion polypeptide is a direct fusion of leptin polypeptide and at least one leptin binding domain of the leptin receptor polypeptide.

25. A nucleic acid molecule comprising a nucleic acid sequence selected from:
i) a nucleic acid sequence as represented in SEQ ID NO:3;
ii) a nucleic acid sequence as represented in SEQ ID NO:5;
iii) a nucleic acid sequence as represented in SEQ ID NO:7;
iv) a nucleic acid sequence as represented in SEQ ID NO:9;
v) a nucleic acid sequence as represented in SEQ ID NO:11;
vi) a nucleic acid sequence as represented in SEQ ID NO:13;
vii) a nucleic acid sequence as represented in SEQ ID NO:15;
viii) a nucleic acid sequence as represented in SEQ ID NO:17;
ix) a nucleic acid sequence as represented in SEQ ID NO:19;
x) a nucleic acid sequence as represented in SEQ ID NO:21;
xi) a nucleic acid sequence as represented in SEQ ID NO:23;
xii) a nucleic acid sequence as represented in SEQ ID NO:25;
xiii) a nucleic acid sequence as represented in SEQ ED NO:27; and
xiv) a nucleic acid sequence that hybridizes under stringent hybridization conditions to SEQ ID NO: 3, 5, 7, 9, 11, 13, 15, 17, 19, 21, 23, 25 or 27 and which encodes a polypeptide that has leptin receptor modulating activity.

26-40. (canceled)

41. An isolated polypeptide encoded by the nucleic acid molecule according to claim 25.

42. An isolated polypeptide comprising an amino acid sequence as represented in SEQ ID NO: 4, 6, 8, 10, 12, 14, 16, 18, 20, 22, 24, 26, 28, 29, 30, 31, 32, 33, 34, 35, 36, 37, 38, 39 or 40.

43. A homodimer consisting of two polypeptides wherein each of said polypeptides comprises:
i) a first part comprising leptin, or a receptor binding domain thereof; and
ii) a second part comprising at least one leptin binding domain or part thereof, of the leptin receptor.

44. A homodimer according to claim 43 wherein said homodimer comprises two polypeptides comprising SEQ ID NO: 2, 4, 6, 8, 10, 12, 14, 16, 18, 20, 22, 24, 26, 28, 29, 30, 31, 32, 33, 34, 35, 36, 37, 38, 39 or 40.

45. A vector comprising a nucleic acid molecule according to claim 25.

46. A cell transfected or transformed with a nucleic acid molecule according to claim 25.

47-48. (canceled)

49. A pharmaceutical composition comprising a polypeptide according to claim 2 and an excipient or carrier.

50. (canceled)

51. A method to treat a human subject suffering from a condition that would benefit from administration of a leptin agonist comprising administering an effective amount of at least one polypeptide according to claim 2.

52. The method according to claim 51 wherein said condition is selected from the group consisting of: obesity; obesity related conditions; type II diabetes; heart disease; and immune suppression.

53-74. (canceled)


SEQUENCE LISTING (48 sequences)

SEQ ID NO: 1 - DNA, 468 bp, artificial
  bacterial expressed leptin
atggtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcctcgagcaccaccaccaccaccactga

SEQ ID NO: 2 - protein, 154 aa, artificial
  bacterial expressed leptin
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCLEHHHHHH

SEQ ID NO: 3 - DNA, 1206 bp, Artificial
  leptin/leptin receptor fusion protein
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcggtggcggaggtagtggtggcggaggtagcggtggcggaggttctggtggcggaggttccggtggcggaggtagtattgatgtcaatatcaatatctcatgtgaaactgatgggtacttaactaaaatgacttgcagatggtcaaccagtacaatccagtcacttgcggaaagcactttgcaattgaggtatcataggagcagcctttactgttctgatattccatctattcatcccatatctgagcccaaagattgctatttgcagagtgatggtttttatgaatgcattttccagccaatcttcctattatctggctacacaatgtggattaggatcaatcactctctaggttcacttgactctccaccaacatgtgtccttcctgattctgtggtgaagccactgcctccatccagtgtgaaagcagaaattactataaacattggattattgaaaatatcttgggaaaagccagtctttccagagaataaccttcaattccagattcgctatggtttaagtggaaaagaagtacaatggaagatgtatgaggtttatgatgcaaaatcaaaatctgtcagtctcccagttccagacttgtgtgcagtctatgctgttcaggtgcgctgtaagaggctagatggactgggatattggagtaattggagcaatccagcctacacagttgtcatggattaatga

SEQ ID NO: 4 - protein, 400 aa, Artificial
  leptin/leptin fusion protein
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMD

SEQ ID NO: 5 - DNA, 1167 bp, Artificial
  leptin/leptin receptor fusion protein bacterial expressed
atggtcccaatccaaaaagttcaagatgacaccaaaaccctgattaagactatcgttacgcgtattaatgacatcagccatacccaaagcgtgtcttcaaaacagaaagtgacaggtctggattttattcctggcctgcatcctatcctgacgctcagtaaaatggatcagacgctcgcggtctatcagcaaattctgacctccatgccgtcccgtaacgtgattcagatttcgaatgatctggaaaatctccgcgatctgctgcacgtattagcattctcaaagtcatgccatttgccgtgggcgagtggtttagaaacgctggatagtctgggtggcgtacttgaagcctccggctatagtaccgaagttgtggccctctctcgcctgcaaggctctttacaggacatgctttggcagctggatctgtctcctgggtgtggcggtgggggttcaggaggcggagggagtgggggcggaggctccggcggagggggctcgggcgggggcggaagcattgatgttaatatcaatattagctgcgaaaccgatggttatttgacgaaaatgacttgccgttggagcacctcaactattcagagtctggccgaatcgacattacagcttcgttatcaccgtagctcgttatattgttctgacatcccaagcatccacccaatttcagaacctaaagattgctatttacagagtgacggtttctatgagtgtatctttcagccgatttttcttctctccggttacaccatgtggattcggatcaaccatagcttgggttcgcttgattcgccgccaacttgtgttttgccagactcagtcgttaaaccgctgccgcctagttccgttaaggctgagattacaatcaacattggcctgttaaaaatcagctgggagaagccggtatttccggaaaacaatcttcagttccaaattcgttacggtttatctggaaaagaagtgcagtggaaaatgtacgaggtatatgatgcaaaatccaagtctgtttctctgccggtcccggatctgtgcgcagtgtatgctgtgcaggtgcgttgtaaacgcctggacggtttgggttactggtcaaactggagcaacccagcgtacacagtagtcatggacctcgagcaccaccaccaccaccactga

SEQ ID NO: 6 - protein, 379 aa, Artificial
  leptin/leptin receptor fusion protein
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMD

SEQ ID NO: 7 - DNA, 3036 bp, Artificial
  leptin/leptin receptor fusion
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcggtggcggaggtagtggtggcggaggtagcggtggcggaggttctggtggcggaggttccggtggcggaggtagttttaacttgtcatatccaattactccttggagatttaagttgtcttgcatgccaccaaattcaacctatgactacttccttttgcctgctggactctcaaagaatacttcaaattcgaatggacattatgagacagctgttgaacctaagtttaattcaagtggtactcacttttctaacttatccaaaacaactttccactgttgctttcggagtgagcaagatagaaactgctccttatgtgcagacaacattgaaggaaagacatttgtttcaacagtaaattctttagtttttcaacaaatagatgcaaactggaacatacagtgctggctaaaaggagacttaaaattattcatctgttatgtggagtcattatttaagaatctattcaggaattataactataaggtccatcttttatatgttctgcctgaagtgttagaagattcacctctggttccccaaaaaggcagttttcagatggttcactgcaattgcagtgttcatgaatgttgtgaatgtcttgtgcctgtgccaacagccaaactcaacgacactctccttatgtgtttgaaaatcacatctggtggagtaattttccagtcacctctaatgtcagttcagcccataaatatggtgaagcctgatccaccattaggtttgcatatggaaatcacagatgatggtaatttaaagatttcttggtccagcccaccattggtaccatttccacttcaatatcaagtgaaatattcagagaattctacaacagttatcagagaagctgacaagattgtctcagctacatccctgctagtagacagtatacttcctgggtcttcgtatgaggttcaggtgaggggcaagagactggatggcccaggaatctggagtgactggagtactcctcgtgtctttaccacacaagatgtcatatactttccacctaaaattctgacaagtgttgggtctaatgtttcttttcactgcatctataagaaggaaaacaagattgttccctcaaaagagattgtttggtggatgaatttagctgagaaaattcctcaaagccagtatgatgttgtgagtgatcatgttagcaaagttacttttttcaatctgaatgaaaccaaacctcgaggaaagtttacctatgatgcagtgtactgctgcaatgaacatgaatgccatcatcgctatgctgaattatatgtgattgatgtcaatatcaatatctcatgtgaaactgatgggtacttaactaaaatgacttgcagatggtcaaccagtacaatccagtcacttgcggaaagcactttgcaattgaggtatcataggagcagcctttactgttctgatattccatctattcatcccatatctgagcccaaagattgctatttgcagagtgatggtttttatgaatgcattttccagccaatcttcctattatctggctacacaatgtggattaggatcaatcactctctaggttcacttgactctccaccaacatgtgtccttcctgattctgtggtgaagccactgcctccatccagtgtgaaagcagaaattactataaacattggattattgaaaatatcttgggaaaagccagtctttccagagaataaccttcaattccagattcgctatggtttaagtggaaaagaagtacaatggaagatgtatgaggtttatgatgcaaaatcaaaatctgtcagtctcccagttccagacttgtgtgcagtctatgctgttcaggtgcgctgtaagaggctagatggactgggatattggagtaattggagcaatccagcctacacagttgtcatggatataaaagttcctatgagaggacctgaattttggagaataattaatggagatactatgaaaaaggagaaaaatgtcactttactttggaagcccctgatgaaaaatgactcattgtgcagtgttcagagatatgtgataaaccatcatacttcctgcaatggaacatggtcagaagatgtgggaaatcacacgaaattcactttcctgtggacagagcaagcacatactgttacggttctggccatcaattcaattggtgcttctgttgcaaattttaatttaaccttttcatggcctatgagcaaagtaaatatcgtgcagtcactcagtgcttatcctttaaacagcagttgtgtgattgtttcctggatactatcacccagtgattacaagctaatgtattttattattgagtggaaaaatcttaatgaagatggtgaaataaaatggcttagaatctcttcatctgttaagaagtattatatccatgatcattttatccccattgagaagtaccagttcagtctttacccaatatttatggaaggagtgggaaaaccaaagataattaatagtttcactcaagatgatattgaaaaacaccagagtgattaatga

SEQ ID NO: 8 - protein, 1010 aa, Artificial
  leptin/leptin receptor fusion
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSFNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSD

SEQ ID NO: 9 - DNA, 3549 bp, Artificial
  leptin/leptin receptor fusion protein
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcggtggcggaggtagtggtggcggaggtagcggtggcggaggttctggtggcggaggttccggtggcggaggtagtgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcggtggcggaggtagtggtggcggaggtagcggtggcggaggttctggtggcggaggttccggtggcggaggtagttttaacttgtcatatccaattactccttggagatttaagttgtcttgcatgccaccaaattcaacctatgactacttccttttgcctgctggactctcaaagaatacttcaaattcgaatggacattatgagacagctgttgaacctaagtttaattcaagtggtactcacttttctaacttatccaaaacaactttccactgttgctttcggagtgagcaagatagaaactgctccttatgtgcagacaacattgaaggaaagacatttgtttcaacagtaaattctttagtttttcaacaaatagatgcaaactggaacatacagtgctggctaaaaggagacttaaaattattcatctgttatgtggagtcattatttaagaatctattcaggaattataactataaggtccatcttttatatgttctgcctgaagtgttagaagattcacctctggttccccaaaaaggcagttttcagatggttcactgcaattgcagtgttcatgaatgttgtgaatgtcttgtgcctgtgccaacagccaaactcaacgacactctccttatgtgtttgaaaatcacatctggtggagtaattttccagtcacctctaatgtcagttcagcccataaatatggtgaagcctgatccaccattaggtttgcatatggaaatcacagatgatggtaatttaaagatttcttggtccagcccaccattggtaccatttccacttcaatatcaagtgaaatattcagagaattctacaacagttatcagagaagctgacaagattgtctcagctacatccctgctagtagacagtatacttcctgggtcttcgtatgaggttcaggtgaggggcaagagactggatggcccaggaatctggagtgactggagtactcctcgtgtctttaccacacaagatgtcatatactttccacctaaaattctgacaagtgttgggtctaatgtttcttttcactgcatctataagaaggaaaacaagattgttccctcaaaagagattgtttggtggatgaatttagctgagaaaattcctcaaagccagtatgatgttgtgagtgatcatgttagcaaagttacttttttcaatctgaatgaaaccaaacctcgaggaaagtttacctatgatgcagtgtactgctgcaatgaacatgaatgccatcatcgctatgctgaattatatgtgattgatgtcaatatcaatatctcatgtgaaactgatgggtacttaactaaaatgacttgcagatggtcaaccagtacaatccagtcacttgcggaaagcactttgcaattgaggtatcataggagcagcctttactgttctgatattccatctattcatcccatatctgagcccaaagattgctatttgcagagtgatggtttttatgaatgcattttccagccaatcttcctattatctggctacacaatgtggattaggatcaatcactctctaggttcacttgactctccaccaacatgtgtccttcctgattctgtggtgaagccactgcctccatccagtgtgaaagcagaaattactataaacattggattattgaaaatatcttgggaaaagccagtctttccagagaataaccttcaattccagattcgctatggtttaagtggaaaagaagtacaatggaagatgtatgaggtttatgatgcaaaatcaaaatctgtcagtctcccagttccagacttgtgtgcagtctatgctgttcaggtgcgctgtaagaggctagatggactgggatattggagtaattggagcaatccagcctacacagttgtcatggatataaaagttcctatgagaggacctgaattttggagaataattaatggagatactatgaaaaaggagaaaaatgtcactttactttggaagcccctgatgaaaaatgactcattgtgcagtgttcagagatatgtgataaaccatcatacttcctgcaatggaacatggtcagaagatgtgggaaatcacacgaaattcactttcctgtggacagagcaagcacatactgttacggttctggccatcaattcaattggtgcttctgttgcaaattttaatttaaccttttcatggcctatgagcaaagtaaatatcgtgcagtcactcagtgcttatcctttaaacagcagttgtgtgattgtttcctggatactatcacccagtgattacaagctaatgtattttattattgagtggaaaaatcttaatgaagatggtgaaataaaatggcttagaatctcttcatctgttaagaagtattatatccatgatcattttatccccattgagaagtaccagttcagtctttacccaatatttatggaaggagtgggaaaaccaaagataattaatagtttcactcaagatgatattgaaaaacaccagagtgattaatga

SEQ ID NO: 10 - protein, 1181 aa, Artificial
  leptin/leptin receptor fusion protein
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSFNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSD

SEQ ID NO: 11 - DNA, 1527 bp, Artificial
  leptin/leptin receptor fusion protein
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcggtggcggaggtagtggtggcggaggtagcggtggcggaggttctggtggcggaggttccggtggcggaggtagtggtggcggaggtagccgtgtctttaccacacaagatgtcatatactttccacctaaaattctgacaagtgttgggtctaatgtttcttttcactgcatctataagaaggaaaacaagattgttccctcaaaagagattgtttggtggatgaatttagctgagaaaattcctcaaagccagtatgatgttgtgagtgatcatgttagcaaagttacttttttcaatctgaatgaaaccaaacctcgaggaaagtttacctatgatgcagtgtactgctgcaatgaacatgaatgccatcatcgctatgctgaattatatgtgattgatgtcaatatcaatatctcatgtgaaactgatgggtacttaactaaaatgacttgcagatggtcaaccagtacaatccagtcacttgcggaaagcactttgcaattgaggtatcataggagcagcctttactgttctgatattccatctattcatcccatatctgagcccaaagattgctatttgcagagtgatggtttttatgaatgcattttccagccaatcttcctattatctggctacacaatgtggattaggatcaatcactctctaggttcacttgactctccaccaacatgtgtccttcctgattctgtggtgaagccactgcctccatccagtgtgaaagcagaaattactataaacattggattattgaaaatatcttgggaaaagccagtctttccagagaataaccttcaattccagattcgctatggtttaagtggaaaagaagtacaatggaagatgtatgaggtttatgatgcaaaatcaaaatctgtcagtctcccagttccagacttgtgtgcagtctatgctgttcaggtgcgctgtaagaggctagatggactgggatattggagtaattggagcaatccagcctacacagttgtcatggattaatga

SEQ ID NO: 12 - protein, 507 aa, Artificial [sequence omitted]

SEQ ID NO: 13 - DNA, 1227 bp, Artificial
  leptin/leptin receptor fusion protein
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcggtggcggaggtagtggtggcggaggtagcggtggcggaggttctggtggcggaggttccggtggcggaggtagtggtggcggaggtagccgtgtctttaccacacaagatgtcatatactttccacctaaaattctgacaagtgttgggtctaatgtttcttttcactgcatctataagaaggaaaacaagattgttccctcaaaagagattgtttggtggatgaatttagctgagaaaattcctcaaagccagtatgatgttgtgagtgatcatgttagcaaagttacttttttcaatctgaatgaaaccaaacctcgaggaaagtttacctatgatgcagtgtactgctgcaatgaacatgaatgccatcatcgctatgctgaattatatgtgattgatgtcaatatcaatatctcatgtgaaactgatgggtacttaactaaaatgacttgcagatggtcaaccagtacaatccagtcacttgcggaaagcactttgcaattgaggtatcataggagcagcctttactgttctgatattccatctattcatcccatatctgagcccaaagattgctatttgcagagtgatggtttttatgaatgcattttccagccaatcttcctattatctggctacacaatgtggattaggatcaatcactctctaggttcacttgactctccaccaacatgtgtccttcctgattctgtggtgtaatga

SEQ ID NO: 14 - protein, 407 aa, Artificial
  leptin/leptin receptor fusion protein
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSGGGGSRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVV

SEQ ID NO: 15 - DNA, 1221 bp, Artificial
  leptin/leptin receptor fusion protein
atgatttgtcaaaaattctgtgtggttttgttacattgggaatttatttatgtgataactgcgattgatgtcaatatcaatatctcatgtgaaactgatgggtacttaactaaaatgacttgcagatggtcaaccagtacaatccagtcacttgcggaaagcactttgcaattgaggtatcataggagcagcctttactgttctgatattccatctattcatcccatatctgagcccaaagattgctatttgcagagtgatggtttttatgaatgcattttccagccaatcttcctattatctggctacacaatgtggattaggatcaatcactctctaggttcacttgactctccaccaacatgtgtccttcctgattctgtggtgaagccactgcctccatccagtgtgaaagcagaaattactataaacattggattattgaaaatatcttgggaaaagccagtctttccagagaataaccttcaattccagattcgctatggtttaagtggaaaagaagtacaatggaagatgtatgaggtttatgatgcaaaatcaaaatctgtcagtctcccagttccagacttgtgtgcagtctatgctgttcaggtgcgctgtaagaggctagatggactgggatattggagtaattggagcaatccagcctacacagttgtcatggatggtggcggaggtagtggtggcggaggtagcggtggcggaggttctggtggcggaggttccggtggcggaggtagtggtggcggaggtagcgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctaatga

SEQ ID NO: 16 - protein, 405 aa, Artificial
  leptin/leptin receptor fusion protein
MICQKFCVVLLHWEFIYVITAIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDGGGGSGGGGSGGGGSGGGGSGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 17 - DNA, 3051 bp, Artificial
  leptin/leptin receptor fusion protein
atgatttgtcaaaaattctgtgtggttttgttacattgggaatttatttatgtgataactgcgtttaacttgtcatatccaattactccttggagatttaagttgtcttgcatgccaccaaattcaacctatgactacttccttttgcctgctggactctcaaagaatacttcaaattcgaatggacattatgagacagctgttgaacctaagtttaattcaagtggtactcacttttctaacttatccaaaacaactttccactgttgctttcggagtgagcaagatagaaactgctccttatgtgcagacaacattgaaggaaagacatttgtttcaacagtaaattctttagtttttcaacaaatagatgcaaactggaacatacagtgctggctaaaaggagacttaaaattattcatctgttatgtggagtcattatttaagaatctattcaggaattataactataaggtccatcttttatatgttctgcctgaagtgttagaagattcacctctggttccccaaaaaggcagttttcagatggttcactgcaattgcagtgttcatgaatgttgtgaatgtcttgtgcctgtgccaacagccaaactcaacgacactctccttatgtgtttgaaaatcacatctggtggagtaattttccagtcacctctaatgtcagttcagcccataaatatggtgaagcctgatccaccattaggtttgcatatggaaatcacagatgatggtaatttaaagatttcttggtccagcccaccattggtaccatttccacttcaatatcaagtgaaatattcagagaattctacaacagttatcagagaagctgacaagattgtctcagctacatccctgctagtagacagtatacttcctgggtcttcgtatgaggttcaggtgaggggcaagagactggatggcccaggaatctggagtgactggagtactcctcgtgtctttaccacacaagatgtcatatactttccacctaaaattctgacaagtgttgggtctaatgtttcttttcactgcatctataagaaggaaaacaagattgttccctcaaaagagattgtttggtggatgaatttagctgagaaaattcctcaaagccagtatgatgttgtgagtgatcatgttagcaaagttacttttttcaatctgaatgaaaccaaacctcgaggaaagtttacctatgatgcagtgtactgctgcaatgaacatgaatgccatcatcgctatgctgaattatatgtgattgatgtcaatatcaatatctcatgtgaaactgatgggtacttaactaaaatgacttgcagatggtcaaccagtacaatccagtcacttgcggaaagcactttgcaattgaggtatcataggagcagcctttactgttctgatattccatctattcatcccatatctgagcccaaagattgctatttgcagagtgatggtttttatgaatgcattttccagccaatcttcctattatctggctacacaatgtggattaggatcaatcactctctaggttcacttgactctccaccaacatgtgtccttcctgattctgtggtgaagccactgcctccatccagtgtgaaagcagaaattactataaacattggattattgaaaatatcttgggaaaagccagtctttccagagaataaccttcaattccagattcgctatggtttaagtggaaaagaagtacaatggaagatgtatgaggtttatgatgcaaaatcaaaatctgtcagtctcccagttccagacttgtgtgcagtctatgctgttcaggtgcgctgtaagaggctagatggactgggatattggagtaattggagcaatccagcctacacagttgtcatggatataaaagttcctatgagaggacctgaattttggagaataattaatggagatactatgaaaaaggagaaaaatgtcactttactttggaagcccctgatgaaaaatgactcattgtgcagtgttcagagatatgtgataaaccatcatacttcctgcaatggaacatggtcagaagatgtgggaaatcacacgaaattcactttcctgtggacagagcaagcacatactgttacggttctggccatcaattcaattggtgcttctgttgcaaattttaatttaaccttttcatggcctatgagcaaagtaaatatcgtgcagtcactcagtgcttatcctttaaacagcagttgtgtgattgtttcctggatactatcacccagtgattacaagctaatgtattttattattgagtggaaaaatcttaatgaagatggtgaaataaaatggcttagaatctcttcatctgttaagaagtattatatccatgatcattttatccccattgagaagtaccagttcagtctttacccaatatttatggaaggagtgggaaaaccaaagataattaatagtttcactcaagatgatattgaaaaacaccagagtgatggtggcggaggtagtggtggcggaggtagcggtggcggaggttctggtggcggaggttccggtggcggaggtagtggtggcggaggtagcgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctaatga

SEQ ID NO: 18 - protein, 1015 aa, Artificial
  leptin/leptin receptor fusion protein
MICQKFCVVLLHWEFIYVITAFNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDGGGGSGGGGSGGGGSGGGGSGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 19 - DNA, 3549 bp, Artificial
  leptin/leptin receptor fusion protein
atgatttgtcaaaaattctgtgtggttttgttacattgggaatttatttatgtgataactgcgtttaacttgtcatatccaattactccttggagatttaagttgtcttgcatgccaccaaattcaacctatgactacttccttttgcctgctggactctcaaagaatacttcaaattcgaatggacattatgagacagctgttgaacctaagtttaattcaagtggtactcacttttctaacttatccaaaacaactttccactgttgctttcggagtgagcaagatagaaactgctccttatgtgcagacaacattgaaggaaagacatttgtttcaacagtaaattctttagtttttcaacaaatagatgcaaactggaacatacagtgctggctaaaaggagacttaaaattattcatctgttatgtggagtcattatttaagaatctattcaggaattataactataaggtccatcttttatatgttctgcctgaagtgttagaagattcacctctggttccccaaaaaggcagttttcagatggttcactgcaattgcagtgttcatgaatgttgtgaatgtcttgtgcctgtgccaacagccaaactcaacgacactctccttatgtgtttgaaaatcacatctggtggagtaattttccagtcacctctaatgtcagttcagcccataaatatggtgaagcctgatccaccattaggtttgcatatggaaatcacagatgatggtaatttaaagatttcttggtccagcccaccattggtaccatttccacttcaatatcaagtgaaatattcagagaattctacaacagttatcagagaagctgacaagattgtctcagctacatccctgctagtagacagtatacttcctgggtcttcgtatgaggttcaggtgaggggcaagagactggatggcccaggaatctggagtgactggagtactcctcgtgtctttaccacacaagatgtcatatactttccacctaaaattctgacaagtgttgggtctaatgtttcttttcactgcatctataagaaggaaaacaagattgttccctcaaaagagattgtttggtggatgaatttagctgagaaaattcctcaaagccagtatgatgttgtgagtgatcatgttagcaaagttacttttttcaatctgaatgaaaccaaacctcgaggaaagtttacctatgatgcagtgtactgctgcaatgaacatgaatgccatcatcgctatgctgaattatatgtgattgatgtcaatatcaatatctcatgtgaaactgatgggtacttaactaaaatgacttgcagatggtcaaccagtacaatccagtcacttgcggaaagcactttgcaattgaggtatcataggagcagcctttactgttctgatattccatctattcatcccatatctgagcccaaagattgctatttgcagagtgatggtttttatgaatgcattttccagccaatcttcctattatctggctacacaatgtggattaggatcaatcactctctaggttcacttgactctccaccaacatgtgtccttcctgattctgtggtgaagccactgcctccatccagtgtgaaagcagaaattactataaacattggattattgaaaatatcttgggaaaagccagtctttccagagaataaccttcaattccagattcgctatggtttaagtggaaaagaagtacaatggaagatgtatgaggtttatgatgcaaaatcaaaatctgtcagtctcccagttccagacttgtgtgcagtctatgctgttcaggtgcgctgtaagaggctagatggactgggatattggagtaattggagcaatccagcctacacagttgtcatggatataaaagttcctatgagaggacctgaattttggagaataattaatggagatactatgaaaaaggagaaaaatgtcactttactttggaagcccctgatgaaaaatgactcattgtgcagtgttcagagatatgtgataaaccatcatacttcctgcaatggaacatggtcagaagatgtgggaaatcacacgaaattcactttcctgtggacagagcaagcacatactgttacggttctggccatcaattcaattggtgcttctgttgcaaattttaatttaaccttttcatggcctatgagcaaagtaaatatcgtgcagtcactcagtgcttatcctttaaacagcagttgtgtgattgtttcctggatactatcacccagtgattacaagctaatgtattttattattgagtggaaaaatcttaatgaagatggtgaaataaaatggcttagaatctcttcatctgttaagaagtattatatccatgatcattttatccccattgagaagtaccagttcagtctttacccaatatttatggaaggagtgggaaaaccaaagataattaatagtttcactcaagatgatattgaaaaacaccagagtgatggtggcggaggtagtggtggcggaggtagcggtggcggaggttctggtggcggaggttccggtggcggaggtagtgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcggtggcggaggtagtggtggcggaggtagcggtggcggaggttctggtggcggaggttccggtggcggaggtagtgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctaatga

SEQ ID NO: 20 - protein, 1181 aa, Artificial
  leptin/leptin receptor fusion protein
MICQKFCVVLLHWEFIYVITAFNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDGGGGSGGGGSGGGGSGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 21 - DNA, 1527 bp, Artificial
  leptin/leptin receptor fusion protein
atgatttgtcaaaaattctgtgtggttttgttacattgggaatttatttatgtgataactgcgcgtgtctttaccacacaagatgtcatatactttccacctaaaattctgacaagtgttgggtctaatgtttcttttcactgcatctataagaaggaaaacaagattgttccctcaaaagagattgtttggtggatgaatttagctgagaaaattcctcaaagccagtatgatgttgtgagtgatcatgttagcaaagttacttttttcaatctgaatgaaaccaaacctcgaggaaagtttacctatgatgcagtgtactgctgcaatgaacatgaatgccatcatcgctatgctgaattatatgtgattgatgtcaatatcaatatctcatgtgaaactgatgggtacttaactaaaatgacttgcagatggtcaaccagtacaatccagtcacttgcggaaagcactttgcaattgaggtatcataggagcagcctttactgttctgatattccatctattcatcccatatctgagcccaaagattgctatttgcagagtgatggtttttatgaatgcattttccagccaatcttcctattatctggctacacaatgtggattaggatcaatcactctctaggttcacttgactctccaccaacatgtgtccttcctgattctgtggtgaagccactgcctccatccagtgtgaaagcagaaattactataaacattggattattgaaaatatcttgggaaaagccagtctttccagagaataaccttcaattccagattcgctatggtttaagtggaaaagaagtacaatggaagatgtatgaggtttatgatgcaaaatcaaaatctgtcagtctcccagttccagacttgtgtgcagtctatgctgttcaggtgcgctgtaagaggctagatggactgggatattggagtaattggagcaatccagcctacacagttgtcatggatggtggcggaggtagtggtggcggaggtagcggtggcggaggttctggtggcggaggttccggtggcggaggtagtggtggcggaggtagcgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctaatga

SEQ ID NO: 22 - protein, 507 aa, Artificial
  leptin/leptin receptor fusion protein
MICQKFCVVLLHWEFIYVITARVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDGGGGSGGGGSGGGGSGGGGSGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 23 - DNA, 1227 bp, Artificial
  leptin/leptin receptor fusion protein
atgatttgtcaaaaattctgtgtggttttgttacattgggaatttatttatgtgataactgcgcgtgtctttaccacacaagatgtcatatactttccacctaaaattctgacaagtgttgggtctaatgtttcttttcactgcatctataagaaggaaaacaagattgttccctcaaaagagattgtttggtggatgaatttagctgagaaaattcctcaaagccagtatgatgttgtgagtgatcatgttagcaaagttacttttttcaatctgaatgaaaccaaacctcgaggaaagtttacctatgatgcagtgtactgctgcaatgaacatgaatgccatcatcgctatgctgaattatatgtgattgatgtcaatatcaatatctcatgtgaaactgatgggtacttaactaaaatgacttgcagatggtcaaccagtacaatccagtcacttgcggaaagcactttgcaattgaggtatcataggagcagcctttactgttctgatattccatctattcatcccatatctgagcccaaagattgctatttgcagagtgatggtttttatgaatgcattttccagccaatcttcctattatctggctacacaatgtggattaggatcaatcactctctaggttcacttgactctccaccaacatgtgtccttcctgattctgtggtgggtggcggaggtagtggtggcggaggtagcggtggcggaggttctggtggcggaggttccggtggcggaggtagtggtggcggaggtagcgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctaatga

SEQ ID NO: 24 - protein, 407 aa, Artificial
  leptin/leptin receptor fusion protein
MICQKFCVVLLHWEFIYVITARVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVGGGGSGGGGSGGGGSGGGGSGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 25 - DNA, 2139 bp, Artificial
  leptin/leptin receptor fusion protein
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcggtggcggaggtagtggtggcggaggtagcggtggcggaggttctggtggcggaggttccggtggcggaggtagtggtggcggaggtagccgtgtctttaccacacaagatgtcatatactttccacctaaaattctgacaagtgttgggtctaatgtttcttttcactgcatctataagaaggaaaacaagattgttccctcaaaagagattgtttggtggatgaatttagctgagaaaattcctcaaagccagtatgatgttgtgagtgatcatgttagcaaagttacttttttcaatctgaatgaaaccaaacctcgaggaaagtttacctatgatgcagtgtactgctgcaatgaacatgaatgccatcatcgctatgctgaattatatgtgattgatgtcaatatcaatatctcatgtgaaactgatgggtacttaactaaaatgacttgcagatggtcaaccagtacaatccagtcacttgcggaaagcactttgcaattgaggtatcataggagcagcctttactgttctgatattccatctattcatcccatatctgagcccaaagattgctatttgcagagtgatggtttttatgaatgcattttccagccaatcttcctattatctggctacacaatgtggattaggatcaatcactctctaggttcacttgactctccaccaacatgtgtccttcctgattctgtggtgaagccactgcctccatccagtgtgaaagcagaaattactataaacattggattattgaaaatatcttgggaaaagccagtctttccagagaataaccttcaattccagattcgctatggtttaagtggaaaagaagtacaatggaagatgtatgaggtttatgatgcaaaatcaaaatctgtcagtctcccagttccagacttgtgtgcagtctatgctgttcaggtgcgctgtaagaggctagatggactgggatattggagtaattggagcaatccagcctacacagttgtcatggatataaaagttcctatgagaggacctgaattttggagaataattaatggagatactatgaaaaaggagaaaaatgtcactttactttggaagcccctgatgaaaaatgactcattgtgcagtgttcagagatatgtgataaaccatcatacttcctgcaatggaacatggtcagaagatgtgggaaatcacacgaaattcactttcctgtggacagagcaagcacatactgttacggttctggccatcaattcaattggtgcttctgttgcaaattttaatttaaccttttcatggcctatgagcaaagtaaatatcgtgcagtcactcagtgcttatcctttaaacagcagttgtgtgattgtttcctggatactatcacccagtgattacaagctaatgtattttattattgagtggaaaaatcttaatgaagatggtgaaataaaatggcttagaatctcttcatctgttaagaagtattatatccatgatcattttatccccattgagaagtaccagttcagtctttacccaatatttatggaaggagtgggaaaaccaaagataattaatagtttcactcaagatgatattgaaaaacaccagagtgattaatga

SEQ ID NO: 26 - protein, 711 aa, Artificial
  leptin/leptin receptor fusion protein
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSGGGGSRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSD

SEQ ID NO: 27 - DNA, 2319 bp, Artificial
  leptin/leptin receptor fusion protein
atgatttgtcaaaaattctgtgtggttttgttacattgggaatttatttatgtgataactgcgggacattatgagacagctgttgaacctaagtttaattcaagtggtactcacttttctaacttatccaaaacaactttccactgttgctttcggagtgagcaagatagaaactgctccttatgtgcagacaacattgaaggaaagacatttgtttcaacagtaaattctttagtttttcaacaaatagatgcaaactggaacatacagtgctggctaaaaggagacttaaaattattcatctgttatgtggagtcattatttaagaatctattcaggaattataactataaggtccatcttttatatgttctgcctgaagtgttagaagattcacctctggttccccaaaaaggcagttttcagatggttcactgcaattgcagtgttcatgaatgttgtgaatgtcttgtgcctgtgccaacagccaaactcaacgacactctccttatgtgtttgaaaatcacatctggtggagtaattttccagtcacctctaatgtcagttcagcccataaatatggtgaagcctgatccaccattaggtttgcatatggaaatcacagatgatggtaatttaaagatttcttggtccagcccaccattggtaccatttccacttcaatatcaagtgaaatattcagagaattctacaacagttatcagagaagctgacaagattgtctcagctacatccctgctagtagacagtatacttcctgggtcttcgtatgaggttcaggtgaggggcaagagactggatggcccaggaatctggagtgactggagtactcctcgtgtctttaccacacaagatgtcatatactttccacctaaaattctgacaagtgttgggtctaatgtttcttttcactgcatctataagaaggaaaacaagattgttccctcaaaagagattgtttggtggatgaatttagctgagaaaattcctcaaagccagtatgatgttgtgagtgatcatgttagcaaagttacttttttcaatctgaatgaaaccaaacctcgaggaaagtttacctatgatgcagtgtactgctgcaatgaacatgaatgccatcatcgctatgctgaattatatgtgattgatgtcaatatcaatatctcatgtgaaactgatgggtacttaactaaaatgacttgcagatggtcaaccagtacaatccagtcacttgcggaaagcactttgcaattgaggtatcataggagcagcctttactgttctgatattccatctattcatcccatatctgagcccaaagattgctatttgcagagtgatggtttttatgaatgcattttccagccaatcttcctattatctggctacacaatgtggattaggatcaatcactctctaggttcacttgactctccaccaacatgtgtccttcctgattctgtggtgaagccactgcctccatccagtgtgaaagcagaaattactataaacattggattattgaaaatatcttgggaaaagccagtctttccagagaataaccttcaattccagattcgctatggtttaagtggaaaagaagtacaatggaagatgtatgaggtttatgatgcaaaatcaaaatctgtcagtctcccagttccagacttgtgtgcagtctatgctgttcaggtgcgctgtaagaggctagatggactgggatattggagtaattggagcaatccagcctacacagttgtcatggatggtggcggaggtagtggtggcggaggtagcggtggcggaggttctggtggcggaggttccggtggcggaggtagtggtggcggaggtagcgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctaatga

SEQ ID NO: 28 - protein, 771 aa, Artificial
  leptin/leptin receptor fusion protein
MICQKFCVVLLHWEFIYVITAGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDGGGGSGGGGSGGGGSGGGGSGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 29 - protein, 379 aa, Artificial; identical to SEQ ID NO: 6
  leptin/leptin receptor fusion protein

SEQ ID NO: 30 - protein, 989 aa, Artificial
  leptin/leptin receptor fusion protein
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSFNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSD

SEQ ID NO: 31 - protein, 1160 aa, Artificial
  leptin/leptin receptor fusion protein
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSFNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSD

SEQ ID NO: 32 - protein, 486 aa, Artificial
  leptin/leptin receptor fusion protein
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSGGGGSRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMD

SEQ ID NO: 33 - protein, 386 aa, Artificial
  leptin/leptin receptor fusion protein
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSGGGGSRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVV

SEQ ID NO: 34 - protein, 384 aa, Artificial
  leptin/leptin receptor fusion
IDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDGGGGSGGGGSGGGGSGGGGSGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 35 - protein, 994 aa, Artificial
  leptin/leptin receptor fusion
FNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDGGGGSGGGGSGGGGSGGGGSGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 36 - protein, 1160 aa, Artificial
  leptin/leptin receptor fusion
FNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDGGGGSGGGGSGGGGSGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 37 - protein, 486 aa, Artificial
  leptin/leptin receptor fusion
RVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDGGGGSGGGGSGGGGSGGGGSGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 38 - protein, 386 aa, Artificial
  leptin/leptin receptor fusion
RVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVGGGGSGGGGSGGGGSGGGGSGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 39 - protein, 690 aa, Artificial
  leptin/leptin receptor fusion
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSGGGGSRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSD

SEQ ID NO: 40 - protein, 750 aa, Artificial
  leptin/leptin receptor fusion
GHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDGGGGSGGGGSGGGGSGGGGSGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 41 - protein, 1165 aa, Homo sapiens
MICQKFCVVLLHWEFIYVITAFNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDAGLYVIVPVIISSSILLLGTLLISHQRMKKLFWEDVPNPKNCSWAQGLNFQKPETFEHLFIKHTASVTCGPLLLEPETISEDISVDTSWKNKDEMMPTTVVSLLSTTDLEKGSVCISDQFNSVNFSEAEGTEVTYEDESQRQPFVKYATLISNSKPSETGEEQGLINSSVTKCFSSKNSPLKDSFSNSSWEIEAQAFFILSDQHPNIISPHLTFSEGLDELLKLEGNFPEENNDKKSIYYLGVTSIKKRESGVLLTDKSRVSCPFPAPCLFTDIRVLQDSCSHFVENNINLGTSSKKTFASYMPQFQTCSTQTHKIMENKMCDLTV

SEQ ID NO: 42 - DNA, 258 bp, Artificial
  vector nucleic acid
aggtggtggccctgagcaggctgcaggggtctctgcaggacatgctgtggcagctggacctcagccctgggtgcggtggcggaggtagtggtggcggaggtagcggtggcggaggttctggtggcggaggttccggtggcggaggtaacttgtcatatccaattactccttggagatttaagttgtcttgcatgccaccaaattcaacctatgactacttccttttgcctgctggactctcaaagaatacttcaaatt

SEQ ID NO: 43 - DNA, 363 bp, Artificial
  vector nucleic acid
aggtggtggccctgagcaggctgcaggggtctctgcaggacatgctgtggcagctggacctcagccctgggtgcggtggcggaggtagtggtggcggaggtagcggtggcggaggttctggtggcggaggttccggtggcggaggtagtattgatgtcaatatcaatatctcatgtgaaactgatgggtacttaactaaaatgacttgcagatggtcaaccagtacaatccagtcacttgcggaaagcactttgcaattgaggtatcataggagcagcctttactgttctgatattccatctattcatcccatatctgagcccaaagattgctatttgcagagtgatggtttttatgaatgca

SEQ ID NO: 44 - protein, 5 aa, Artificial
  peptide linking molecule
GGGGS

SEQ ID NO: 45 - DNA, 38 bp, Artificial
  leptin forward oligonucleotide primer
gggaaagctagccaccatgcattggggaaccctgtgcg

SEQ ID NO: 46 - DNA, 31 bp, Artificial
  leptin reverse primer
gggaaaggatccgcacccagggctgaggtcc

SEQ ID NO: 47 - DNA, 38 bp, Artificial; identical to SEQ ID NO: 45
  leptin receptor oligonucleotide primer

SEQ ID NO: 48 - DNA, 31 bp, Artificial; identical to SEQ ID NO: 46
  leptin receptor oligonucleotide primer